SEQUENCE LISTING (66 sequences)

SEQ ID NO: 1 - protein, 463 aa, Artificial sequence
  Modified Homo sapiens immunoglobulin G1 heavy chain
MNLLLILTFVAAAVAQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSV

SEQ ID NO: 3 - protein, 234 aa, Artificial sequence
  Modified Homo sapiens immunoglobulin G1 kappa light chain
MNLLLILTFVAAAVADVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Mus musculus
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKR

SEQ ID NO: 5 - protein, 327 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 6 - protein, 415 aa, Equus caballus
SLEDTAVIPLFSECKAPKEDDVVSLACLVKGYFPEPVQVTWEPEMQNQKPWTFPAMKKGQEYIHVFSLTTWWKPGSHSCTVHHKASSFRKKMTFQEPASWAPQRTSALPVTSKEPTPAPTTLRKSEPSTRHTQPETQKPRIPVDTPLKECQSHTHPPSIYLLHPPLQGLWLKGEATFTCLVVGDDLKDAHLSWELSERSNGMFVESGPLEKHTNGSQSRSSRLALPRSSWAMGTSVTCKLSYPNLLSSMEVVGLKEHAASAPRSLTVHALTTPGLNASPGATSWLQCKVSGFSPPEIVLTWLEGQREVDPSWFATARPTAQPGNTTFQTWSILLVPTIPGPPTATYTCVVGHEASRQLLNTSWSLDTGGLAMTPESKDENSDDYADLDDAGSLWLTFMALFLITLLYSGFVTFIK

SEQ ID NO: 7 - protein, 334 aa, Canis familiaris
SKTSPSVFPLSLCHQESEGYVVIGCLVQGFFPPEPVNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSNASLTCTLSGLKDPKGATFTWNPSKGKEPIQKNPERDSCGCYSVSSVLPGCADPWNHGDTFSCTATHPESKSPITVSITKTTEHIPPQVHLLPPPSEELALNELVTLTCLVRGFKPKDVLVRWLQGTQELPQEKYLTWEPLKEPDQTNMFAVTSMLRVTAEDWKQGEKFSCMVGHEALPMSFTQKTIDRLAGKPTHVNVSVV

SEQ ID NO: 8 - protein, 426 aa, Canis familiaris
TSQDLSVFPLASCCKDNIASTSVTLGCLVTGYLPMSTTVTWDTGSLNKNVTTFPTTFHETYGLHSIVSQVTASGKWAKQRFTCSVAHAESTAINKTFSACALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 9 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 10 - protein, 96 aa, Camelus dromedarius
EVQLVESGGGLVQPGGSLRLSCAASGFTFSSYDMSWVRQAPGREREGVAAINSGGGSTYYADSVKGRFTISQDNAKNTVYLQMNSLKPEDTAMYYC

SEQ ID NO: 11 - protein, 96 aa, Camelus dromedarius
EVQLVESGGGLVQPGGSLRLSCAASGFTFSSYWMYWVRQAPGKGLEWVSTINSGGGSTYYADSVKGRFTISRDNAKNMLYLQMNSLKPEDTAMYYC

SEQ ID NO: 12 - DNA, 1434 bp, Artificial sequence
  Murine anti-AGE IgG2b heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctaagaccacccctccctccgtgtaccctctggctcctggctgtggcgacaccaccggatcctctgtgaccctgggctgcctcgtgaagggctacttccctgagtccgtgaccgtgacctggaactccggctccctgtcctcctccgtgcacacctttccagccctgctgcagtccggcctgtacaccatgtcctccagcgtgacagtgccctcctccacctggccttcccagaccgtgacatgctctgtggcccaccctgcctcttccaccaccgtggacaagaagctggaaccctccggccccatctccaccatcaacccttgccctccctgcaaagaatgccacaagtgccctgcccccaacctggaaggcggcccttccgtgttcatcttcccacccaacatcaaggacgtgctgatgatctccctgacccccaaagtgacctgcgtggtggtggacgtgtccgaggacgaccctgacgtgcagatcagttggttcgtgaacaacgtggaagtgcacaccgcccagacccagacacacagagaggactacaacagcaccatcagagtggtgtctaccctgcccatccagcaccaggactggatgtccggcaaagaattcaagtgcaaagtgaacaacaaggacctgcccagccccatcgagcggaccatctccaagatcaagggcctcgtgcgggctccccaggtgtacattctgcctccaccagccgagcagctgtcccggaaggatgtgtctctgacatgtctggtcgtgggcttcaaccccggcgacatctccgtggaatggacctccaacggccacaccgaggaaaactacaaggacaccgcccctgtgctggactccgacggctcctacttcatctactccaagctgaacatgaagacctccaagtgggaaaagaccgactccttctcctgcaacgtgcggcacgagggcctgaagaactactacctgaagaaaaccatctcccggtcccccggctag

SEQ ID NO: 13 - DNA, 1416 bp, Artificial sequence
  Chimeric anti-AGE human IgG1 antibody heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 14 - DNA, 720 bp, Artificial sequence
  Murine anti-AGE Kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 15 - DNA, 720 bp, Artificial sequence
  Chimeric anti-AGE human kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 16 - protein, 477 aa, Artificial sequence
  Murine anti-AGE IgG2b heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 17 - protein, 471 aa, Artificial sequence
  Chimeric anti-AGE human IgG1 heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 239 aa, Artificial sequence
  Murine anti-AGE kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 239 aa, Artificial sequence
  Chimeric anti-AGE human kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, Artificial sequence; identical to SEQ ID NO: 2
  Murine anti-AGE IgG2b heavy chain (variable region)

SEQ ID NO: 21 - protein, 112 aa, Artificial sequence
  Murine anti-AGE kappa light chain (variable region)
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIK

SEQ ID NO: 22 - protein, 326 aa, Artificial sequence
  Human constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 23 - protein, 7 aa, Artificial sequence
  CDR1H (heavy chain)
SYTMGVS

SEQ ID NO: 24 - protein, 17 aa, Artificial sequence
  CDR2H (heavy chain)
TISSGGGSTYYPDSVKG

SEQ ID NO: 25 - protein, 10 aa, Artificial sequence
  CDR3H (heavy chain)
  misc_feature (10)..(10): Xaa can be any naturally occurring amino acid
QGGWLPPFAX

SEQ ID NO: 26 - protein, 17 aa, Artificial sequence
  CDR1L (light chain)
RASKSVSTSSRGYSYMH

SEQ ID NO: 27 - protein, 7 aa, Artificial sequence
  CDR2L (light chain)
LVSNLES

SEQ ID NO: 28 - protein, 9 aa, Artificial sequence
  CDR3L (light chain)
QHIRELTRS

SEQ ID NO: 29 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNQKFQGRVTMTVDKSTNTVYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaacagaaattccagggcagagtgaccatgaccgtggacaagtccaccaacaccgtgtacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 31 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSTNTAYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccaccaacaccgcttacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 33 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSINTAYMELSRLRSDDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccatcaacaccgcttacatggaactgtccagactgcggagcgatgacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 35 - protein, 238 aa, Artificial Sequence
  Humanized light chain
METDTLLLWVLLLWVPGSTGDVVMTQSPLSLPVTLGQPASISCRSSQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatcctcccagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 37 - protein, 238 aa, Artificial Sequence
  Humanized light chain
METDTLLLWVLLLWVPGSTGDVVMTQSPLSLPVTLGQPASISCRSRQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatccaggcagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 39 - protein, 238 aa, Artificial Sequence
  Humanized light chain
METDTLLLWVLLLWVPGSTGDVVMTQSPLSSPVTLGQPASISCRSSQSLVNSNGNTFLQWYHQRPGQPPRLLIYKVSLRFSGVPDRFSGSGAGKDFTLKISRVEAEDVGVYYCSQSTHVPPTFGQGTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 715 bp, Artificial Sequence
  Humanized light chain
atggagaccgacaccctgctgctctgggtgctgctgctctgggtgcccggctccaccggagacgtcgtgatgacccagtcccctctgtccagtcctgtgaccctgggacagcctgcctccatctcctcagatcctcccagtccctcgtgaactccaacggcaacaccttcctgcagtggtatcaccagcggcctggccagcctcccagactgctgatctacaaggtgtccctgcggttctccggcgtgcccgacgattttccggctctggcgctggcaaggacttcaccctgaagatctcccgggtggaagccgaggacgtgggcgtgtactactgctcccagagcacccacgtgccccctacatttggccagggcaccaactggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 41 - protein, 5 aa, Mus musculus
TYWMH

SEQ ID NO: 42 - protein, 17 aa, Mus musculus
EISPTNGRAYYNARFKS

SEQ ID NO: 43 - protein, 9 aa, Mus musculus
AYGNYEFAY

SEQ ID NO: 44 - protein, 16 aa, Mus musculus
RSRQSLVNSNGNTFLQ

SEQ ID NO: 45 - protein, 7 aa, Mus musculus
KVSLRFS

SEQ ID NO: 46 - protein, 9 aa, Mus musculus
SQSTHVPPT

SEQ ID NO: 47 - protein, 467 aa, Mus musculus
MGWTLVFLFLLSVTAGVHSQVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 467 aa, Artificial sequence
  Humanized heavy chain
MGWTLVFLFLLSVTAGVHSEVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 467 aa, Artificial sequence
  Humanized heavy chain
MGWTLVFLFLLSVTAGVHSQVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 467 aa, Artificial sequence
  Humanized heavy chain
MGWTLVFLFLLSVTAGVHSQVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 467 aa, Artificial sequence
  Humanized heavy chain
MGWTLVFLFLLSVTAGVHSQVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSV

SEQ ID NO: 53 - protein, 118 aa, Artificial sequence
  Humanized heavy chain variable region
EVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSS

SEQ ID NO: 54 - protein, 118 aa, Artificial sequence
  Humanized heavy chain variable region
QVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSS

SEQ ID NO: 55 - protein, 118 aa, Artificial sequence
  Humanized heavy chain variable region
QVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSS

SEQ ID NO: 56 - protein, 118 aa, Artificial sequence
  Humanized heavy chain variable region
QVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSS

SEQ ID NO: 57 - protein, 239 aa, Mus musculus
MVSSAQFLGLLLLCFQGTRCDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 239 aa, Artificial sequence
  Humanized light chain
MVSSAQFLGLLLLCFQGTRCDIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 239 aa, Artificial sequence
  Humanized light chain
MVSSAQFLGLLLLCFQGTRCDIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 239 aa, Artificial sequence
  Humanized light chain
MVSSAQFLGLLLLCFQGTRCDVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 239 aa, Artificial sequence
  Humanized light chain
MVSSAQFLGLLLLCFQGTRCDIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 112 aa, Mus musculus; identical to SEQ ID NO: 21

SEQ ID NO: 63 - protein, 112 aa, Artificial sequence
  Humanized light chain variable region
DIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIK

SEQ ID NO: 64 - protein, 112 aa, Artificial sequence
  Humanized light chain variable region
DIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVK

SEQ ID NO: 65 - protein, 112 aa, Artificial sequence
  Humanized light chain variable region
DVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIK

SEQ ID NO: 66 - protein, 112 aa, Artificial sequence
  Humanized light chain variable region
DIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIK

(19) United States
(12) Patent Application Publication
(10) Pub. No.: US 2024/0000930 A1
(43) Pub. Date: Jan. 4, 2024

(54) METHODS AND COMPOSITIONS FOR TREATING KIDNEY DISEASES

(71) Applicant: Siwa Corporation, Chicago, IL (US)

(72) Inventor: Lewis S. Gruber, Chicago, IL (US)

(21) Appl. No.: 18/265,205

(22) PCT Filed: Dec. 9, 2021
(86) PCT No.: PCT/US2021/062606

§ 371 Date: Jun. 2, 2023

Related U.S. Application Data
(60) Provisional application No. 63/123,326, filed on Dec. 9, 2020.

(51) Int. Cl. A61K 39/395; A61K 39/00; A61P 13/12

(52) U.S. Cl. CPC A61K 39/3955; A61K 39/0005; A61P 13/12

(57) ABSTRACT

A method of treating or preventing the onset of kidney disease comprises administering to a subject a composition comprising an anti-AGE antibody. The anti-AGE antibody binds an AGE antigen comprising at least one protein or peptide that exhibits AGE modifications selected from the group consisting of FFI, pyrraline, AFGP, ALI, carboxymethyllysine, carboxyethyllysine and pentosidine. A method of treating or preventing the onset of kidney disease comprises administering to a subject a vaccine comprising an AGE antigen.

### BACKGROUND

The kidneys are organs that filter blood to remove wastes and excess fluids. The kidneys are composed of about one million filtering units called nephrons, which each include a renal corpuscle, to filter blood, and a tubule, to remove waste and return the filtered blood to the body. The renal corpuscle includes a tuft of capillaries known as the glomerulus surrounded by a sac known as Bowman's capsule. The glomerulus is the primary site of blood filtration, and the functioning of the kidneys may be determined by measuring the glomerular filtration rate (GFR) or the estimated glomerular filtration rate (eGFR). A decreased GFR or eGFR may be a sign of kidney disease.

Kidney diseases, also referred to as renal diseases or nephropathy, reduce the normal functioning of the kidneys and are the ninth leading cause of death in the United States (“Chronic Kidney Disease Basics”, Centers for Disease Control and Prevention, available online at www.cdc.gov/kidneydisease/basics.html (2020)). Kidney diseases may be broadly classified as nephritis if the disease is inflammatory or nephrosis if the disease is non-inflammatory. The most prevalent kidney disease is chronic kidney disease (CKD), which affects about 10% of the population worldwide (“Chronic Kidney Disease”, World Kidney Day, available online at worldkidneyday.org/facts/chronic-kidney-disease/(2020)). Other kidney diseases include diabetic nephropathy, diabetic kidney disease, IgA nephropathy (also referred to as Berger's disease or synpharyngitic glomerulonephritis), Goodpasture's syndrome (also referred to as anti-glomerular basement membrane disease), minimal change disease, thin basement membrane disease, fibronectin glomerulopathy, nephrotic syndrome, Alport syndrome (hereditary nephritis), familial renal amyloidosis, lupus nephritis, Finnish-type nephrosis, acquired cystic kidney disease, pyelonephritis (kidney infection), medullary sponge kidney (Cacchi-Ricci disease) and polycystic kidney disease.

Kidney diseases may be classified based on which part of the kidney they affect. For example, renal tubular diseases include acute tubular necrosis, renal tubular acidosis, polycystic kidney disease, Fanconi syndrome, Bartter syndrome, Gitelman syndrome and Liddle's syndrome. Similarly, interstitial nephritis is inflammation of the interstitial spaces between renal tubules.

Diseases of the glomerulus are of particular concern due to the role of glomeruli in blood filtration. Inflammatory diseases of the glomerulus are generally known as glomerulonephritis (including acute proliferative glomerulonephritis, endocapillary nephritis, mesangioproliferative glomerulonephritis, mesangiocapillary glomerulonephritis, membranoproliferative glomerulonephritis, acute crescentic glomerulonephritis, focal segmental glomerulonephritis, membranous glomerulonephritis, post-infectious glomerulonephritis, acute post-streptococcal glomerulonephritis and rapidly progressive glomerulonephritis). The irreversible formation of fibrotic tissue, such as scarring or hardening, in the glomerulus is referred to as glomerulosclerosis (including focal segmental glomerulosclerosis or FSGS, diabetic glomerulosclerosis and nodular glomerulosclerosis). Glomerular diseases allow waste products to accumulate in the blood and can alter the proper level of proteins and red blood cells in the bloodstream.

Kidney diseases may be managed with lifestyle changes, such as staying physically active and eating a healthy diet. Kidney diseases that occur as a side effect of a separate condition may improve or resolve after treating the underlying condition, such as by managing diabetes with insulin or taking medication to reduce high blood pressure. Kidney diseases may progress to end-stage renal disease (ESRD, also known as end-stage kidney disease or ESKD) or kidney failure, in which the kidneys lose their ability to function properly and can no longer filter wastes from blood. Kidney failure requires regular dialysis to cleanse the blood, typically in the form of hemodialysis or peritoneal dialysis. Kidney transplantation is an option for individuals that are healthy enough for the procedure and who are able to find a suitable donor. Pharmacotherapies for treating or preventing kidney disease are limited and primarily focus on controlling symptoms, reducing complications and slowing the progression of the disease.

Senescent cells are cells that are partially-functional or non-functional and are in a state of proliferative arrest. Senescence is a distinct state of a cell, and is associated with biomarkers, such as activation of the biomarker p16^{Ink4a}, and expression of β-galactosidase. Senescence begins with damage or stress (such as overstimulation by growth factors) of cells.

Advanced glycation end-products (AGEs; also referred to as AGE-modified proteins or peptides, or glycation end-products) arise from a non-enzymatic reaction of sugars with protein side-chains (Ando, K. et aL, Membrane Proteins of Human Erythrocytes Are Modified by Advanced Glycation End Products during Aging in the Circulation, Biochem Biophys Res Commun., Vol. 258, 123, 125 (1999)). This process begins with a reversible reaction between the reducing sugar and the amino group to form a Schiff base, which proceeds to form a covalently-bonded Amadori rearrangement product. Once formed, the Amadori product undergoes further rearrangement to produce AGEs. Hyperglycemia and oxidative stress promote this post-translational modification of membrane proteins (Lindsey J B, et aL, “Receptor For Advanced Glycation End-Products (RAGE) and soluble RAGE (sRAGE): Cardiovascular Implications,” Diabetes Vascular Disease Research, Vol. 6(1), 7-14, (2009)). AGEs may also be formed from other processes. For example, the advanced glycation end product, N^{ε}-(carboxymethyl)lysine, is a product of both lipid peroxidation and glycoxidation reactions. AGEs have been associated with several pathological conditions including inflammation, atherosclerosis, stroke, endothelial cell dysfunction, and neurodegenerative disorders (Bierhaus A, “AGEs and their interaction with AGE-receptors in vascular disease and diabetes mellitus. I. The AGE concept,” Cardiovasc Res, Vol. 37(3), 586-600 (1998)).

AGE-modified proteins are also a marker of senescent cells. This association between AGEs and senescence is well known in the art. See, for example, Gruber, L. (WO 2009/143411, 26 November 2009), Ando, K. et al. (Membrane Proteins of Human Erythrocytes Are Modified by Advanced Glycation End Products during Aging in the Circulation, Biochem Biophys Res Commun., Vol. 258, 123, 125 (1999)), Ahmed, E. K. etal. (“Protein Modification and Replicative Senescence of WI-38 Human Embryonic Fibroblasts” Aging Cells, vol. 9, 252, 260 (2010)), Vlassara, H. et al. (Advanced Glycosylation Endproducts on Erythrocyte Cell Surface Induce Receptor-Mediated Phagocytosis by Macrophages, J. Exp. Med., Vol. 166, 539, 545 (1987)) and Vlassara et al. (“High-affinity-receptor-mediated Uptake and Degradation of Glucose-modified Proteins: A Potential Mechanism for the Removal of Senescent Macromolecules” Proc. Natl. Acad. Sci. USAI, Vol. 82, 5588, 5591 (1985)). Furthermore, Ahmed, E. K. et al. indicates that glycation end-products are “one of the major causes of spontaneous damage to cellular and extracellular proteins” (Ahmed, E. K. et al., see above, page 353). Accordingly, the accumulation of glycation end-products is associated with senescence and lack of function.

The damage or stress that causes cellular senescence also negatively impacts mitochondrial DNA in the cells to cause them to produce free radicals which react with sugars in the cell to form methyl glyoxal (MG). MG in turn reacts with proteins or lipids to generate advanced glycation end products. In the case of the protein component lysine, MG reacts to form carboxymethyllysine, which is an AGE.

Damage or stress to mitochondrial DNA also sets off a DNA damage response which induces the cell to produce cell cycle blocking proteins. These blocking proteins prevent the cell from dividing. Continued damage or stress causes mTOR production, which in turn activates protein synthesis and inactivates protein breakdown. Further stimulation of the cells leads to programmed cell death (apoptosis).

p16 is a protein involved in regulation of the cell cycle, by inhibiting the S phase (synthesis phase). It can be activated during ageing or in response to various stresses, such as DNA damage, oxidative stress or exposure to drugs. p16 is typically considered a tumor suppressor protein, causing a cell to become senescent in response to DNA damage and irreversibly preventing the cell from entering a hyperproliferative state. However, there has been some ambiguity in this regard, as some tumors show overexpression of p16, while others show downregulated expression. Evidence suggests that overexpression of p16 is some tumors results from a defective retinoblastoma protein (“Rb”). p16 acts on Rb to inhibit the S phase, and Rb downregulates p16, creating negative feedback. Defective Rb fails to both inhibit the S phase and downregulate p16, thus resulting in overexpression of p16 in hyperproliferating cells (Romagosa, C. et al., p16^{Ink4a} overexpression in cancer: a tumor suppressor gene associated with senescence and high-grade tumors, Oncogene, Vol. 30, 2087-2097 (2011)).

Senescent cells are associated with secretion of many factors involved in intercellular signaling, including pro-inflammatory factors; secretion of these factors has been termed the senescence-associated secretory phenotype, or SASP (Freund, A. “Inflammatory networks during cellular senescence: causes and consequences” Trends Mol Med. 2010 May;16(5):238-46). Autoimmune diseases, such as Crohn's disease and rheumatoid arthritis, are associated with chronic inflammation (Ferraccioli, G. et al. “Interleukin-1β and Interleukin-6 in Arthritis Animal Models: Roles in the Early Phase of Transition from Acute to Chronic Inflammation and Relevance for Human Rheumatoid Arthritis” Mol Med. 2010 November-December; 16(11-12): 552-557). Chronic inflammation may be characterized by the presence of pro-inflammatory factors at levels higher than baseline near the site of pathology, but lower than those found in acute inflammation. Examples of these factors include TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin. Senescent cells also upregulate genes with roles in inflammation including IL-1β, IL-8, ICAM1, TNFAP3, ESM1 and CCL2 (Burton, D. G. A. et al., “Microarray analysis of senescent vascular smooth muscle cells: a link to atherosclerosis and vascular calcification”, Experimental Gerontology, Vol. 44, No. 10, pp. 659-665 (October 2009)). Because senescent cells produce pro-inflammatory factors, removal of these cells alone produces a profound reduction in inflammation as well as the amount and concentration of pro-inflammatory factors.

Senescent cells secrete reactive oxygen species (“ROS”) as part of the SASP. ROS are believed to play an important role in maintaining senescence of cells. The secretion of ROS creates a bystander effect, where senescent cells induce senescence in neighboring cells: ROS create the very cellular damage known to activate p16 expression, leading to senescence (Nelson, G., A senescent cell bystander effect: senescence-induced senescence, Aging Cell, Vo. 11, 345-349 (2012)). The p16/Rb pathway leads to the induction of ROS, which in turn activates the protein kinase C delta creating a positive feedback loop that further enhance ROS, helping maintain the irreversible cell cycle arrest; it has even been suggested that exposing cancer cells to ROS might be effective to treat cancer by inducing cell phase arrest in hyperproliferating cells (Rayess, H. et at, Cellular senescence and tumor suppressor gene p16, Int J Cancer, Vol. 130, 1715-1725 (2012)).

Recent research demonstrates the therapeutic benefits of removing senescent cells. In vivo animal studies at the Mayo Clinic in Rochester, Minnesota, found that elimination of senescent cells in transgenic mice carrying a biomarker for elimination delayed age-related disorders associated with cellular senescence. Eliminating senescent cells in fat and muscle tissues substantially delayed the onset of sarcopenia and cataracts and reduced senescence indicators in skeletal muscle and the eye (Baker, D. J. et al., “Clearance of p16^{Ink4a}-positive senescent cells delays ageing-associated disorders”, Nature, Vol. 479, pp. 232-236, (2011)). Mice that were treated to induce senescent cell elimination were found to have larger diameters of muscle fibers as compared to untreated mice. Treadmill exercise tests indicated that treatment also preserved muscle function. Continuous treatment of transgenic mice for removal of senescent cells had no negative side effects and selectively delayed age-related phenotypes that depend on cells. This data demonstrates that removal of senescent cells produces beneficial therapeutic effects and shows that these benefits may be achieved without adverse effects.

Additional In vivo animal studies in mice found that removing senescent cells using senolytic agents treats aging-related disorders and atherosclerosis. Short-term treatment with senolytic drugs in chronologically aged or progeroid mice alleviated several aging-related phenotypes (Zhu, Y. et al., “The Achilles’ heel of senescent cells: from transcriptome to senolytic drugs”, Aging Cell, vol. 14, pp. 644-658 (2015)). Long-term treatment with senolytic drugs improved vasomotor function in mice with established atherosclerosis and reduced intimal plaque calcification (Roos, C. M. et al., “Chronic senolytic treatment alleviates established vasomotor dysfunction in aged or atherosclerotic mice”, Aging Cell (2016)). This data further demonstrates the benefits of removing senescent cells.

Vaccines have been widely used since their introduction by Edward Jenner in the 1770s to confer immunity against a wide range of diseases and afflictions. Vaccine preparations contain a selected immunogenic agent capable of stimulating immunity to an antigen. Typically, antigens are used as the immunogenic agent in vaccines, such as, for example, viruses, either killed or attenuated, and purified viral components. Antigens used in the production of cancer vaccines include, for example, tumor-associated carbohydrate antigens (TACAs), dendritic cells, whole cells and viral vectors. Different techniques are employed to produce the desired amount and type of antigen being sought. For example, pathogenic viruses are grown either in eggs or cells. Recombinant DNA technology is often utilized to generate attenuated viruses for vaccines.

Vaccines may therefore be used to stimulate the production of antibodies in the body and provide immunity against antigens. When an antigen is introduced to a subject that has been vaccinated and developed immunity to that antigen, the immune system may destroy or remove cells that express the antigen.

### SUMMARY

In a first aspect, the invention is a method of treating or preventing the onset of kidney disease comprising administering to a subject a composition comprising an anti-AGE antibody.

In a second aspect, the invention is a method of treating or preventing the onset of kidney disease comprising administering to a subject a vaccine comprising an AGE antigen.

### DEFINITIONS

The terms “kidney disease”, “renal disease” and “nephropathy” may be used interchangeably to refer to a disease or disorder that reduces the functioning of the kidneys. Examples of kidney diseases include chronic kidney disease (CKD), diabetic nephropathy, diabetic kidney disease, IgA nephropathy (also referred to as Berger's disease or synpharyngitic glomerulonephritis), Goodpasture's syndrome (also referred to as anti-glomerular basement membrane disease), minimal change disease, thin basement membrane disease, fibronectin glomerulopathy, nephrotic syndrome, Alport syndrome (hereditary nephritis), familial renal amyloidosis, lupus nephritis, Finnish-type nephrosis, acquired cystic kidney disease, pyelonephritis (kidney infection), medullary sponge kidney (Cacchi-Ricci disease), polycystic kidney disease, acute tubular necrosis, renal tubular acidosis, polycystic kidney disease, Fanconi syndrome, Bartter syndrome, Gitelman syndrome, Liddle's syndrome, interstitial nephritis, glomerulonephritis (including acute proliferative glomerulonephritis, endocapillary nephritis, mesangioproliferative glomerulonephritis, mesangiocapillary glomerulonephritis, membranoproliferative glomerulonephritis, acute crescentic glomerulonephritis, focal segmental glomerulonephritis, membranous glomerulonephritis, post-infectious glomerulonephritis, acute post-streptococcal glomerulonephritis and rapidly progressive glomerulonephritis) and glomerulosclerosis (including focal segmental glomerulosclerosis or FSGS, diabetic glomerulosclerosis and nodular glomerulosclerosis).

The term “peptide” means a molecule composed of 2-50 amino acids.

The term “protein” means a molecule composed of more than 50 amino acids.

The terms “advanced glycation end-product”, “AGE”, “AGE-modified protein”, “AGE-modified peptide” and “glycation end-product” refer to modified proteins or peptides that are formed as the result of the reaction of sugars with protein side chains that further rearrange and form irreversible cross-links. This process begins with a reversible reaction between a reducing sugar and an amino group to form a Schiff base, which proceeds to form a covalently-bonded Amadori rearrangement product. Once formed, the Amadori product undergoes further rearrangement to produce AGEs. AGE-modified proteins and antibodies to AGE-modified proteins are described in U.S. Pat. No. 5,702,704 to Bucala (“Bucala”) and U.S. Pat. No. 6,380,165 to Al-Abed et al. (“Al-Abed”). Glycated proteins or peptides that have not undergone the necessary rearrangement to form AGEs, such as N-deoxyfructosyllysine found on glycated albumin, are not AGEs. AGEs may be identified by the presence of AGE modifications (also referred to as AGE epitopes or AGE moieties) such as 2-(2-furoyl)-4(5)-(2-furanyl)-1H-imidazole (“FFI”); 5-hydroxymethyl-1-alkylpyrrole-2-carbaldehyde (“Pyrraline”); 1-alkyl-2-formyl-3,4-diglycosyl pyrrole (“AFGP”), a non-fluorescent model AGE; carboxymethyllysine; carboxyethyllysine; and pentosidine. ALI, another AGE, is described in Al-Abed.

The term “AGE antigen” means a substance that elicits an immune response against an AGE-modified protein or peptide of a cell. The immune response against an AGE-modified protein or peptide of a cell does not include the production of antibodies to the non-AGE-modified protein or peptide.

“An antibody that binds to an AGE-modified protein on a cell”, “anti-AGE antibody” or “AGE antibody” means an antibody, antibody fragment or other protein or peptide that binds to an AGE-modified protein or peptide which preferably includes a constant region of an antibody, where the protein or peptide which has been AGE-modified is a protein or peptide normally found bound on the surface of a cell, preferably a mammalian cell, more preferably a human, cat, dog, horse, camelid (for example, camel or alpaca), cattle, sheep, or goat cell. “An antibody that binds to an AGE-modified protein on a cell”, “anti-AGE antibody” or “AGE antibody” does not include an antibody or other protein which binds with the same specificity and selectivity to both the AGE-modified protein or peptide, and the same non-AGE-modified protein or peptide (that is, the presence of the AGE modification does not increase binding). AGE-modified albumin is not an AGE-modified protein on a cell, because albumin is not a protein normally found bound on the surface of cells. “An antibody that binds to an AGE-modified protein on a cell”, “anti-AGE antibody” or “AGE antibody” only includes those antibodies which lead to removal, destruction, or death of the cell. Also included are antibodies which are conjugated, for example to a toxin, drug, or other chemical or particle. Preferably, the antibodies are monoclonal antibodies, but polyclonal antibodies are also possible.

The term “senescent cell” means a cell which is in a state of proliferative arrest and expresses one or more biomarkers of senescence, such as activation of p16^{Ink4a} or expression of senescence-associated β-galactosidase. Also included are cells which express one or more biomarkers of senescence, do not proliferate in vivo, but may proliferate in vitro under certain conditions, such as some satellite cells found in the muscles of ALS patients.

The term “variant” means a nucleotide, protein or amino acid sequence different from the specifically identified sequences, wherein one or more nucleotides, proteins or amino acid residues is deleted, substituted or added. Variants may be naturally-occurring allelic variants, or non-naturally-occurring variants. Variants of the identified sequences may retain some or all of the functional characteristics of the identified sequences.

The term “percent (%) sequence identity” is defined as the percentage of amino acid residues in a candidate sequence that are identical to the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using publicly available computer software such as BLAST, BLAST-2, ALIGN or Megalign (DNASTAR) software. Preferably, % sequence identity values are generated using the sequence comparison computer program ALIGN-2. The ALIGN-2 sequence comparison computer program is publicly available from Genentech, Inc. (South San Francisco, CA), or may be compiled from the source code, which has been filed with user documentation in the U.S. Copyright Office and is registered under U.S. Copyright Registration No. TXU510087. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained using the ALIGN-2 computer program.

### BRIEF DESCRIPTION OF THE DRAWING

FIG. 1 is a graph of the response versus time in an antibody binding experiment.

FIG. 2 is a graph of the count of senescent cells and various concentrations of hydrogen peroxide.

### DETAILED DESCRIPTION

Kidney disease has long been viewed as a side effect of other diseases and disorders, particularly diabetes, systemic lupus erythematosus, hypertension and infections such as human immunodeficiency virus (HIV) and bacterial endocarditis. Kidney disease is also a known side effect of long-term analgesic use and exposure to substances such as iodinated contrast media, lithium and chemotherapeutics. Recent research has revealed that cellular senescence is a main proponent in the development and progression of various kidney diseases.

Senescent cells have been directly implicated in kidney diseases such as chronic kidney disease (CKD). Cellular senescence and the SASP participate in the pathological process of CKD, and CKD accelerates the progression of cellular senescence and the secretion of inflammatory factors through the SASP (Wang, W-J. et al., “Cellular senescence, senescence-associated secretory phenotype, and chronic kidney disease”, Oncotarget, Vol. 8, No. 38, pp. 64520-64533 (2017)). Much like the SASP, CKD is characterized by oxidative stress and chronic low-grade inflammation, and this inflammatory state has been referred to as the CKD-associated secretory phenotype or CASP. CKD involves a significant increase in the expression of the senescence markers senescence-associated β-galactosidase (SA-(β-gal) and p16 protein in the glomeruli, tubules and interstitium.

Senescent cells have also been linked to glomerular diseases. Senescent cells increase in the glomeruli in response to renal injury and aging (Valentijn, F. A. et al., “Cellular senescence in the aging and diseased kidney”, Journal of Cell Communication and Signaling, Vol. 12, pp. 69-82 (2018)). Increased senescent markers have been detected in various glomerular diseases, including glomerulosclerosis, membranous nephropathy, minimal change disease, IgA nephropathy, focal segmental glomerulosclerosis, and glomerulonephritis (Valentijn, F. A. et al.). In addition, the expression of the senescence marker SA-β-gal was associated with development of glomerular lesions in a mouse model of lupus nephritis (Yang, C. et al., “Accelerated glomerular cell senescence in experimental lupus nephritis”, Medical Science Monitor, Vol. 24, pp. 6882-6891 (2018)). Thus, cellular senescence is a factor in glomerulosclerosis and other kidney diseases.

The relationship between AGEs and kidney disease also implicates cellular senescence since AGEs are recognized as a marker of senescent cells. AGEs play a major role in kidney disease by promoting oxidative stress and inflammation, and are markedly increased in CKD (Uribarri, J. et al., “The low AGE diet: a neglected aspect of clinical nephrology practice?”, Nephron, Vol. 130, pp. 48-53 (2015)). AGEs cause ageing of the renal tubular epithelial cells and increase their expression of the senescence markers SA-β-gal and p16 in CKD (Wang, W-J. et al.). Levels of carboxymethyllysine (CML), the most well-known AGE, in the serum are independently associated with CKD and estimated glomerular filtration rate (eGFR), a measure of kidney function (Semba, R. D. et al., “Serum carboxymethyl-lysine, a dominant advanced glycation end product, is associated with chronic kidney disease: the Baltimore longitudinal study of aging”, Journal of Renal Nutrition, Vol. 20, No. 2, pp. 74-81 (2010)). AGEs, including CML, were significantly higher in IgA nephropathy patients with decreased renal function as comparted to similar patients with normal renal function (Vas, T. et al., “Oxidative stress and non-enzymatic glycation in IgA nephropathy”, Clinical Nephrology, Vol. 64, No. 5, pp. 343-351 (2005)). AGE exposure followed by binding to RAGE results in tubulointerstitial fibrosis in the diabetic kidney (Oldfield, M. D. et al., “Advanced glycation end products cause epithelial-myofibroblast transdifferentiation via the receptor for advanced glycation end products (RAGE)”, The Journal of Clinical Investigation, Vol. 108, No. 12, pp. 1853-1863 (2001)). AGEs can be one of the major factors influencing the progression of diabetic nephropathy by modulating the expression of matrix metalloproteinases (Xu, X., etal., “A glimpse of matrix metalloproteinases in diabetic nephropathy”, Current Medicinal Chemistry, Vol. 21, No. 28, pp. 3244-3260 (2014)).

AGEs have also been linked to glomerular diseases. Levels of CML were significantly increased in the glomeruli in a diabetic rat model, suggesting that AGEs may be one of the major causes of diabetic glomerulosclerosis (Kushiro, M. et al., “Accumulation of N^{σ}-(carboxy-methyl)lysine and changes in glomerular extracellular matrix components in Otsuka Long-Evans Tokushima fatty rat: a model of spontaneous NIDDM”, Nephron, Vol. 79, No. 4, pp. 458-468 (1998)). Multiple studies have shown that the AGEs CML and pentosidine accumulate in the glomeruli of diabetic nephropathy patients (Horie, K. et al., “Immunohistochemical colocalization of glycoxidation products and lipid peroxidation products in diabetic renal glomerular lesions”, The Journal of Clinical Investigation, Vol. 100, No. 12, pp. 2995-3004 (1997), Suzuki, D. et al., “Immunohistochemical evidence for an increased oxidative stress and carbonyl modification of proteins in diabetic glomerular lesions”, Journal of the American Society of Nephrology, Vol. 10, pp. 822-832 (1999)). CML and pentosidine have also been identified in glomerulosclerosis, FSGS, hypertensive nephrosclerosis and lupus nephritis (Tanji, N. et al., “Expression of advanced glycation end products and their cellular receptor RAGE in diabetic nephropathy and nondiabetic renal disease”, Journal of the American Society of Nephrology, Vol. 11, pp. 1656-1666 (2000)). AGEs have been shown to cross-link proteins in the kidney extracellular matrix and affect glomerular endothelial cells in vitro (Uribarri, J. et al.). Accordingly, AGEs, especially CML and pentosidine, are associated with glomerulosclerosis and other kidney diseases.

The identification of a link between AGEs and kidney disease has resulted in research into therapies that target AGEs to treat kidney disease. Reducing the level of serum AGEs has been shown to improve kidney function. A low-AGE diet almost completely abrogated kidney disease in a diabetic mouse model (Uribarri, J. et al.). AGE inhibitors such as aminoguanidine, benfotiamine, pyridoxamine, OPB-9195, and AGE breakers have all been shown to ameliorate diabetic nephropathy in diabetic rats (Uribarri, J. et al.). The AGE inhibitor aminoguanidine also reduces signs of diabetic nephropathy in the glomeruli (Horie, K. et al.). Interfering with AGE-RAGE binding with anti-RAGE or anti-TGF-β antibodies or the AGE breaker ALT711 reduced tubulointerstitial fibrosis (Oldfield, M. D. et al.). These studies suggest that targeting AGEs is an appropriate treatment for kidney diseases.

Similarly, previous studies have found that removal of senescent cells improves kidney function. Clearance of senescent cells by induction of apoptosis in transgenic animal models attenuated glomerulosclerosis (Valentijn, F. A. et al.). In addition, the administration of senolytic agents preserved kidney function during aging and reduced age-associated damage to the kidney (Valentijn, F. A. et al.). These studies indicate that removal of senescent cells is a viable treatment for kidney diseases including glomerulosclerosis.

The therapeutic benefits of removing senescent cells has been demonstrated in vivo in an art-accepted model in treating age-related diseases such as sarcopenia (U.S. Pat. No. 9,161,810) and treating metastatic cancer (WO 2017/143073). The identification of a link between cellular senescence, either directly or through AGEs, and kidney disease allows for similar treatment possibilities. The present invention uses enhanced clearance of cells expressing AGE-modified proteins or peptides (AGE-modified cells) to treat, ameliorate or prevent the onset of kidney disease by removing or killing senescent cells. This may be accomplished by administering anti-AGE antibodies to a subject.

Vaccination against AGE-modified proteins or peptides of a cell may also be used to control the presence of AGE-modified cells in a subject. The continuous and virtually ubiquitous surveillance exercised by the immune system in the body in response to a vaccination allows maintaining low levels of AGE-modified cells in the body. Vaccination against AGE-modified proteins or peptides of a cell removes or kills senescent cells. The process of senescent cell removal or destruction allows vaccination against AGE-modified proteins or peptides of a cell to be used to treat or prevent the onset of kidney disease.

An antibody that binds to an AGE-modified protein on a cell (“anti-AGE antibody” or “AGE antibody”) is known in the art. Examples include those described in U.S. Pat. No. 5,702,704 (Bucala) and U.S. Pat. No. 6,380,165 (Al-Abed et al.). The antibody may bind to one or more AGE-modified proteins or peptides having an AGE modification such as FFI, pyrraline, AFGP, ALI, carboxymethyllysine, carboxyethyllysine and pentosidine, and mixtures of such antibodies. Preferably, the antibody binds carboxymethyllysine-modified or carboxyethyllysine-modified proteins. Preferably, the antibody is non-immunogenic to the animal in which it will be used, such as non-immunogenic to humans; companion animals including cats, dogs and horses; and commercially important animals, such camels (or alpaca), cattle (bovine), sheep, and goats. More preferably, the antibody has the same species constant region as antibodies of the animal to reduce the immune response against the antibody, such as being humanized (for humans), felinized (for cats), caninized (for dogs), equuinized (for horses), camelized (for camels or alpaca), bovinized (for cattle), ovinized (for sheep), or caperized (for goats). Most preferably, the antibody is identical to that of the animal in which it will be used (except for the variable region), such as a human antibody, a cat antibody, a dog antibody, a horse antibody, a camel antibody, a bovine antibody, a sheep antibody or a goat antibody. Details of the constant regions and other parts of antibodies for these animals are described below. The antibody may be monoclonal or polyclonal. Preferably, the antibody is a monoclonal antibody.

Preferred anti-AGE antibodies include those which bind to proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents). A particularly preferred antibody includes the variable region of the commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, MN; catalog no. MAB3247), modified to have a human constant region (or the constant region of the animal into which it will be administered). Commercially-available antibodies, such as the carboxymethyl lysine antibody corresponding to catalog no. MAB3247 from R&D Systems, Inc., may be intended for diagnostic purposes and may contain material that is not suited for use in animals or humans. Preferably, commercially-available antibodies are purified and/or isolated prior to use in animals or humans to remove toxins or other potentially-harmful material.

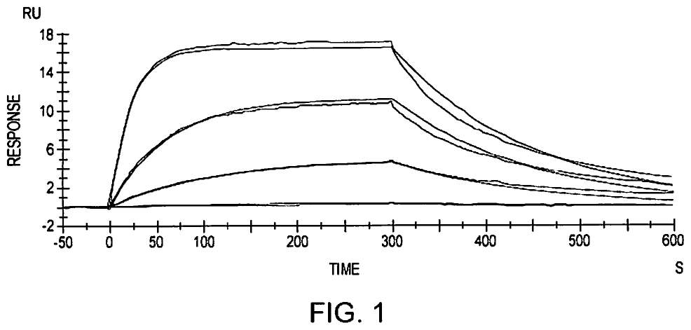

The anti-AGE antibody has low rate of dissociation from the antibody-antigen complex, or k_{d} (also referred to as k_{back} or off-rate), preferably at most 9×10⁻³, 8×10⁻³, 7×10⁻³or 6×10⁻³ (sec⁻¹). The anti-AGE antibody has a high affinity for the AGE-modified protein of a cell, which may be expressed as a low dissociation constant K_{D} of at most 9×10⁻⁶, 8×10⁻⁶, 7×10⁻⁶, 6×10⁻⁶, 5×10⁻⁶, 4×10⁻⁶ or 3×10⁻⁶ (M). Preferably, the binding properties of the anti-AGE antibody are similar to, the same as, or superior to the carboxymethyl lysine MAb (Clone 318003) available from R&D Systems, Inc. (Minneapolis, MN; catalog no. MAB3247), illustrated in FIG. 1.

The anti-AGE antibody may destroy AGE-modified cells through antibody-dependent cell-mediated cytotoxicity (ADCC). ADCC is a mechanism of cell-mediated immune defense in which an effector cell of the immune system actively lyses a target cell whose membrane-surface antigens have been bound by specific antibodies. ADCC may be mediated by natural killer (NK) cells, macrophages, neutrophils or eosinophils. The effector cells bind to the Fc portion of the bound antibody. The anti-AGE antibody may also destroy AGE-modified cells through complement-dependent cytotoxicity (CDC). In CDC, the complement cascade of the immune system is triggered by an antibody binding to a target antigen.

The anti-AGE antibody may be conjugated to an agent that causes the destruction of AGE-modified cells. Such agents may be a toxin, a cytotoxic agent, magnetic nanoparticles, and magnetic spin-vortex discs.

A toxin, such as pore-forming toxins (PFT) (Aroian R. et al., “Pore-Forming Toxins and Cellular Non-Immune Defenses (CNIDs),” Current Opinion in Microbiology, 10:57-61 (2007)), conjugated to an anti-AGE antibody may be injected into a patient to selectively target and remove AGE-modified cells. The anti-AGE antibody recognizes and binds to AGE-modified cells. Then, the toxin causes pore formation at the cell surface and subsequent cell removal through osmotic lysis.

Magnetic nanoparticles conjugated to the anti-AGE antibody may be injected into a patient to target and remove AGE-modified cells. The magnetic nanoparticles can be heated by applying a magnetic field in order to selectively remove the AGE-modified cells.

As an alternative, magnetic spin-vortex discs, which are magnetized only when a magnetic field is applied to avoid self-aggregation that can block blood vessels, begin to spin when a magnetic field is applied, causing membrane disruption of target cells. Magnetic spin-vortex discs, conjugated to anti-AGE antibodies specifically target AGE-modified cell types, without removing other cells.

Antibodies are Y-shaped proteins composed of two heavy chains and two light chains. The two arms of the Y shape form the fragment antigen-binding (Fab) region while the base or tail of the Y shape forms the fragment crystallizable (Fc) region of the antibody. Antigen binding occurs at the terminal portion of the fragment antigen-binding region (the tips of the arms of the Y shape) at a location referred to as the paratope, which is a set of complementarity determining regions (also known as CDRs or the hypervariable region). The complementarity determining regions vary among different antibodies and gives a given antibody its specificity for binding to a given antigen. The fragment crystallizable region of the antibody determines the result of antigen binding and may interact with the immune system, such as by triggering the complement cascade or initiating antibody-dependent cell-mediated cytotoxicity (ADCC). When antibodies are prepared recombinantly, it is also possible to have a single antibody with variable regions (or complementary determining regions) that bind to two different antigens, with each tip of the Y shape being specific to one of the antigens; these are referred to as bi-specific antibodies.

A humanized anti-AGE antibody according to the present invention may have the human constant region sequence of amino acids shown in SEQ ID NO: 22. The heavy chain complementarity determining regions of the humanized anti-AGE antibody may have one or more of the protein sequences shown in SEQ ID NO: 23 (CDR1H), SEQ ID NO: 24 (CDR2H) and SEQ ID NO: 25 (CDR3H). The light chain complementarity determining regions of the humanized anti-AGE antibody may have one or more of the protein sequences shown in SEQ ID NO: 26 (CDR1L), SEQ ID NO: 27 (CDR2L) and SEQ ID NO: 28 (CDR3L).

The heavy chain of a humanized anti-AGE antibody may have or may include the protein sequence of SEQ ID NO: 1. The variable domain of the heavy chain may have or may include the protein sequence of SEQ ID NO: 2. The complementarity determining regions of the variable domain of the heavy chain (SEQ ID NO: 2) are shown in SEQ ID NO: 41, SEQ ID NO: 42 and SEQ ID NO: 43. The kappa light chain of a humanized anti-AGE antibody may have or may include the protein sequence of SEQ ID NO: 3. The variable domain of the kappa light chain may have or may include the protein sequence of SEQ ID NO: 4. Optionally, the arginine (Arg or R) residue at position 128 of SEQ ID NO: 4 may be omitted. The complementarity determining regions of the variable domain of the light chain (SEQ ID NO: 4) are shown in SEQ ID NO: 44, SEQ ID NO: 45 and SEQ ID NO: 46. The variable regions may be codon-optimized, synthesized and cloned into expression vectors containing human immunoglobulin G1 constant regions. In addition, the variable regions may be used in the preparation of non-human anti-AGE antibodies.

The antibody heavy chain may be encoded by the DNA sequence of SEQ ID NO: 12, a murine anti-AGE immunoglobulin G2b heavy chain. The protein sequence of the murine anti-AGE immunoglobulin G2b heavy chain encoded by SEQ ID NO: 12 is shown in SEQ ID NO: 16. The variable region of the murine antibody is shown in SEQ ID NO: 20, which corresponds to positions 25-142 of SEQ ID NO: 16. The antibody heavy chain may alternatively be encoded by the DNA sequence of SEQ ID NO: 13, a chimeric anti-AGE human immunoglobulin G1 heavy chain. The protein sequence of the chimeric anti-AGE human immunoglobulin G1 heavy chain encoded by SEQ ID NO: 13 is shown in SEQ ID NO: 17. The chimeric anti-AGE human immunoglobulin includes the murine variable region of SEQ ID NO: 20 in positions 25-142. The antibody light chain may be encoded by the DNA sequence of SEQ ID NO: 14, a murine anti-AGE kappa light chain. The protein sequence of the murine anti-AGE kappa light chain encoded by SEQ ID NO: 14 is shown in SEQ ID NO: 18. The variable region of the murine antibody is shown in SEQ ID NO: 21, which corresponds to positions 21-132 of SEQ ID NO: 18. The antibody light chain may alternatively be encoded by the DNA sequence of SEQ ID NO: 15, a chimeric anti-AGE human kappa light chain. The protein sequence of the chimeric anti-AGE human kappa light chain encoded by SEQ ID NO: 15 is shown in SEQ ID NO: 19. The chimeric anti-AGE human immunoglobulin includes the murine variable region of SEQ ID NO: 21 in positions 21-132.

A humanized anti-AGE antibody according to the present invention may have or may include one or more humanized heavy chains or humanized light chains. A humanized heavy chain may be encoded by the DNA sequence of SEQ ID NO: 30, 32 or 34. The protein sequences of the humanized heavy chains encoded by SEQ ID NOs: 30, 32 and 34 are shown in SEQ ID NOs: 29, 31 and 33, respectively. A humanized light chain may be encoded by the DNA sequence of SEQ ID NO: 36, 38 or 40. The protein sequences of the humanized light chains encoded by SEQ ID NOs: 36, 38 and 40 are shown in SEQ ID NOs: 35, 37 and 39, respectively. Preferably, the humanized anti-AGE antibody maximizes the amount of human sequence while retaining the original antibody specificity. A complete humanized antibody may be constructed that contains a heavy chain having a protein sequence chosen from SEQ ID NOs: 29, 31 and 33 and a light chain having a protein sequence chosen from SEQ ID NOs: 35, 37 and 39.

Particularly preferred anti-AGE antibodies may be obtained by humanizing murine monoclonal anti-AGE antibodies. Murine monoclonal anti-AGE antibodies have the heavy chain protein sequence shown in SEQ ID NO: 47 (the protein sequence of the variable domain is shown in SEQ ID NO: 52) and the light chain protein sequence shown in SEQ ID NO: 57 (the protein sequence of the variable domain is shown in SEQ ID NO: 62). A preferred humanized heavy chain may have the protein sequence shown in SEQ ID NO: 48, SEQ ID NO: 49, SEQ ID NO: 50 or SEQ ID NO: 51 (the protein sequences of the variable domains of the humanized heavy chains are shown in SEQ ID NO: 53, SEQ ID NO: 54, SEQ ID NO: 55 and SEQ ID NO: 56, respectively). A preferred humanized light chain may have the protein sequence shown in SEQ ID NO: 58, SEQ ID NO: 59, SEQ ID NO: 60 or SEQ ID NO: 61 (the protein sequences of the variable domains of the humanized light chains are shown in SEQ ID NO: 63, SEQ ID NO: 64, SEQ ID NO: 65 and SEQ ID NO: 66, respectively). Preferably, a humanized anti-AGE monoclonal antibody is composed a heavy chain having a protein sequence selected from the group consisting of SEQ ID NO: 48, SEQ ID NO: 49, SEQ ID NO: 50 and SEQ ID NO: 51 and a light chain having a protein sequence selected from the group consisting of SEQ ID NO: 58, SEQ ID NO: 59, SEQ ID NO: 60 and SEQ ID NO: 61. Humanized monoclonal anti-AGE antibodies composed of these protein sequences may have better binding and/or improved activation of the immune system, resulting in greater efficacy.

The protein sequence of an antibody from a non-human species may be modified to include the variable domain of the heavy chain having the sequence shown in SEQ ID NO: 2 or the kappa light chain having the sequence shown in SEQ ID NO: 4. The non-human species may be a companion animal, such as the domestic cat or domestic dog, or livestock, such as cattle, the horse or the camel. Preferably, the non-human species is not the mouse. The heavy chain of the horse (Equus caballus) antibody immunoglobulin gamma 4 may have or may include the protein sequence of SEQ ID NO: 5 (EMBL/GenBank accession number AY445518). The heavy chain of the horse (Equus caballus) antibody immunoglobulin delta may have or may include the protein sequence of SEQ ID NO: 6 (EMBL/GenBank accession number AY631942). The heavy chain of the dog (Canis familiaris) antibody immunoglobulin A may have or may include the protein sequence of SEQ ID NO: 7 (GenBank accession number L36871). The heavy chain of the dog (Canis familiaris) antibody immunoglobulin E may have or may include the protein sequence of SEQ ID NO: 8 (GenBank accession number L36872). The heavy chain of the cat (Fells catus) antibody immunoglobulin G2 may have or may include the protein sequence of SEQ ID NO: 9 (DDBJ/EMBL/GenBank accession number KF811175).

Animals of the camelid family, such as camels (Camelus dromedarius and Camelus bactrianus), llamas (Lama glama, Lama pacos and Lama vicugna), alpacas (Vicugna pacos) and guanacos (Lama guanicoe), have a unique antibody that is not found in other mammals. In addition to conventional immunoglobulin G antibodies composed of heavy and light chain tetramers, camelids also have heavy chain immunoglobulin G antibodies that do not contain light chains and exist as heavy chain dimers. These antibodies are known as heavy chain antibodies, HCAbs, single-domain antibodies or sdAbs, and the variable domain of a camelid heavy chain antibody is known as the VHH. The camelid heavy chain antibodies lack the heavy chain CH1 domain and have a hinge region that is not found in other species. The variable region of the Arabian camel (Camelus dromedarius) single-domain antibody may have or may include the protein sequence of SEQ ID NO: 10 (GenBank accession number AJ245148). The variable region of the heavy chain of the Arabian camel (Camelus dromedarius) tetrameric immunoglobulin may have or may include the protein sequence of SEQ ID NO: 11 (GenBank accession number AJ245184).

In addition to camelids, heavy chain antibodies are also found in cartilaginous fishes, such as sharks, skates and rays. This type of antibody is known as an immunoglobulin new antigen receptor or IgNAR, and the variable domain of an IgNAR is known as the VNAR. The IgNAR exists as two identical heavy chain dimers composed of one variable domain and five constant domains each. Like camelids, there is no light chain.

The protein sequences of additional non-human species may be readily found in online databases, such as the International ImMunoGeneTics Information System (www.imgt.org), the European Bioinformatics Institute (www.ebi.ac.uk), the DNA Databank of Japan (ddbj.nig.ac.jp/arsa) or the National Center for Biotechnology Information (www.ncbi.nlm.nih.gov).

An anti-AGE antibody or a variant thereof may include a heavy chain having at least 90%, 91%, 92%, 93%, 94%, 95%, 96%, 97%, 98%, 99%, or 100% sequence identity to the amino acid sequence of SEQ ID NO: 1, SEQ ID NO: 16, SEQ ID NO: 17, SEQ ID NO: 29, SEQ ID NO: 31, SEQ ID NO: 33, SEQ ID NO: 47, SEQ ID NO: 48, SEQ ID NO: 49, SEQ ID NO: 50 or SEQ ID NO: 51, including post-translational modifications thereof. A heavy chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE.

An anti-AGE antibody or a variant thereof may include a heavy chain variable region having at least 90%, 91%, 92%, 93%, 94%, 95%, 96%, 97%, 98%, 99%, or 100% sequence identity to the amino acid sequence of SEQ ID NO: 2, SEQ ID NO: 20, SEQ ID NO: 23, SEQ ID NO: 24, SEQ ID NO: 25, SEQ ID NO: 41, SEQ ID NO: 42, SEQ ID NO: 43, SEQ ID NO: 52, SEQ ID NO: 53, SEQ ID NO: 54, SEQ ID NO: 55, or SEQ ID NO: 56, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain having at least 90%, 91%, 92%, 93%, 94%, 95%, 96%, 97%, 98%, 99%, or 100% sequence identity to the amino acid sequence of SEQ ID NO: 3, SEQ ID NO: 18, SEQ ID NO: 19, SEQ ID NO: 35, SEQ ID NO: 37, SEQ ID NO: 39, SEQ ID NO: 57, SEQ ID NO: 58, SEQ ID NO: 59, SEQ ID NO: 60 or SEQ ID NO: 61, including post-translational modifications thereof. A light chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain variable region having at least 90%, 91%, 92%, 93%, 94%, 95%, 96%, 97%, 98%, 99%, or 100% sequence identity to the amino acid sequence of SEQ ID NO: 4, SEQ ID NO: 21, SEQ ID NO: 26, SEQ ID NO: 27, SEQ ID NO: 28, SEQ ID NO: 44, SEQ ID NO: 45, SEQ ID NO: 46, SEQ ID NO: 62, SEQ ID NO: 63, SEQ ID NO: 64, SEQ ID NO: 65 or SEQ ID NO: 66, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

Alternatively, the antibody may have the complementarity determining regions of commercially available mouse anti-glycation end-product antibody raised against carboxymethyl lysine conjugated with keyhole limpet hemocyanin (CML-KLH), the carboxymethyl lysine MAb (Clone 318003) available from R&D Systems, Inc. (Minneapolis, MN; catalog no. MAB3247).

The antibody may have or may include constant regions which permit destruction of targeted cells by a subject's immune system.

Mixtures of antibodies that bind to more than one type AGE of AGE-modified proteins may also be used.

Bi-specific antibodies, which are anti-AGE antibodies directed to two different epitopes, may also be used. Such antibodies will have a variable region (or complementary determining region) from those of one anti-AGE antibody, and a variable region (or complementary determining region) from a different antibody.

Antibody fragments may be used in place of whole antibodies. For example, immunoglobulin G may be broken down into smaller fragments by digestion with enzymes. Papain digestion cleaves the N-terminal side of inter-heavy chain disulfide bridges to produce Fab fragments. Fab fragments include the light chain and one of the two N-terminal domains of the heavy chain (also known as the Fd fragment). Pepsin digestion cleaves the C-terminal side of the inter-heavy chain disulfide bridges to produce F(ab′)₂ fragments. F(ab′)₂ fragments include both light chains and the two N-terminal domains linked by disulfide bridges. Pepsin digestion may also form the Fv (fragment variable) and Fc (fragment crystallizable) fragments. The Fv fragment contains the two N-terminal variable domains. The Fc fragment contains the domains which interact with immunoglobulin receptors on cells and with the initial elements of the complement cascade. Pepsin may also cleave immunoglobulin G before the third constant domain of the heavy chain (C_{H}3) to produce a large fragment F(abc) and a small fragment pFc′. Antibody fragments may alternatively be produced recombinantly. Preferably, such antibody fragments are conjugated to an agent that causes the destruction of AGE-modified cells.

If additional antibodies are desired, they can be produced using well-known methods. For example, polyclonal antibodies (pAbs) can be raised in a mammalian host by one or more injections of an immunogen, and if desired, an adjuvant. Typically, the immunogen (and adjuvant) is injected in a mammal by a subcutaneous or intraperitoneal injection. The immunogen may be an AGE-modified protein of a cell, such as AGE-antithrombin III, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexokinase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-hexokinase, AGE-apo C-I, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Examples of adjuvants include Freund's complete, monophosphoryl Lipid A synthetic-trehalose dicorynomycolate, aluminum hydroxide (alum), heat shock proteins HSP 70 or HSP96, squalene emulsion containing monophosphoryl lipid A, α2-macroglobulin and surface active substances, including oil emulsions, pleuronic polyols, polyanions and dinitrophenol. To improve the immune response, an immunogen may be conjugated to a polypeptide that is immunogenic in the host, such as keyhole limpet hemocyanin (KLH), serum albumin, bovine thyroglobulin, cholera toxin, labile enterotoxin, silica particles or soybean trypsin inhibitor. A preferred immunogen conjugate is AGE-KLH. Alternatively, pAbs may be made in chickens, producing IgY molecules.

Monoclonal antibodies (mAbs) may also be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Other techniques may be used, such as the EBV-hybridoma technique. Techniques for the generation of chimeric antibodies by splicing genes encoding the variable domains of antibodies to genes of the constant domains of human (or other animal) immunoglobulin result in “chimeric antibodies” that are substantially human (humanized) or substantially “ized” to another animal (such as cat, dog, horse, camel or alpaca, cattle, sheep, or goat) at the amino acid level. If desired, the mAbs may be purified from the culture medium or ascites fluid by conventional procedures, such as protein A-sepharose, hydroxyapatite chromatography, gel electrophoresis, dialysis, ammonium sulfate precipitation or affinity chromatography. Additionally, human monoclonal antibodies can be generated by immunization of transgenic mice containing a third copy IgG human trans-loci and silenced endogenous mouse Ig loci or using human-transgenic mice. Production of humanized monoclonal antibodies and fragments thereof can also be generated through phage display technologies.

A “pharmaceutically acceptable carrier” includes any and all solvents, dispersion media, coatings, antibacterial and antifungal agents, isotonic and absorption delaying agents, and the like, compatible with pharmaceutical administration. Preferred examples of such carriers or diluents include water, saline, Ringer's solutions and dextrose solution. Supplementary active compounds can also be incorporated into the compositions. Solutions and suspensions used for parenteral administration can include a sterile diluent, such as water for injection, saline solution, polyethylene glycols, glycerin, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The antibodies may be administered by injection, such as by intravenous injection or locally, such as by intra-articular injection into a joint. Pharmaceutical compositions suitable for injection include sterile aqueous solutions or dispersions for the extemporaneous preparation of sterile injectable solutions or dispersion. Various excipients may be included in pharmaceutical compositions of antibodies suitable for injection. Suitable carriers include physiological saline, bacteriostatic water, CREMOPHOR EL® (BASF; Parsippany, NJ) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin. Sterile injectable solutions can be prepared by incorporating antibodies, and optionally other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

For administration by inhalation, the antibodies may be delivered as an aerosol spray from a nebulizer or a pressurized container that contains a suitable propellant, for example, a gas such as carbon dioxide. Antibodies may also be delivered via inhalation as a dry powder, for example using the iSPERSE™ inhaled drug delivery platform (PULMATRIX, Lexington, Mass.). The use of anti-AGE antibodies which are chicken antibodies (IgY) may be non-immunogenic in a variety of animals, including humans, when administered by inhalation.

An appropriate dosage level of each type of antibody will generally be about to 500 mg per kg patient body weight. Preferably, the dosage level will be about 0.1 to about 250 mg/kg; more preferably about 0.5 to about 100 mg/kg. A suitable dosage level may be about 0.01 to 250 mg/kg, about 0.05 to 100 mg/kg, or about 0.1 to 50 mg/kg. Within this range the dosage may be 0.05 to 0.5, 0.5 to 5 or to 50 mg/kg. Although each type of antibody may be administered on a regimen of 1 to 4 times per day, such as once or twice per day, antibodies typically have a long half-life in vivo. Accordingly, each type of antibody may be administered once a day, once a week, once every two or three weeks, once a month, or once every 60 to 90 days.

A subject that receives administration of an anti-AGE antibody may be tested to determine if the administration has been effective to treat kidney disease. Kidney disease may be monitored by measuring glomerular filtration rate (GFR) or estimated glomerular filtration rate (eGFR), urinalysis to detect protein or red blood cells in the urine, blood tests to measure waste products such as creatinine and urea nitrogen, renal imaging such as ultrasound or X-rays or a kidney biopsy. A preferred method of monitoring kidney disease is GFR or eGFR. A subject may be considered to have received an effective antibody treatment if he or she demonstrates an increase in GFR or eGFR between subsequent measurements or over time. Alternatively, the concentration and/or number of senescent cells may be measured over time. Administration of antibody and subsequent testing may be repeated until the desired therapeutic result is achieved.

Unit dosage forms can be created to facilitate administration and dosage uniformity. Unit dosage form refers to physically discrete units suited as single dosages for the subject to be treated, containing a therapeutically effective quantity of one or more types of antibodies in association with the required pharmaceutical carrier. Preferably, the unit dosage form is in a sealed container and is sterile.

Vaccines against AGE-modified proteins or peptides contain an AGE antigen, an adjuvant, optional preservatives and optional excipients. Examples of AGE antigens include AGE-modified proteins or peptides such as AGE-antithrombin III, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexokinase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-hexokinase, AGE-apo C-I, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Suitable AGE antigens also include proteins or peptides that exhibit AGE modifications (also referred to as AGE epitopes or AGE moieties) such as carboxymethyllysine (CML), carboxyethyllysine (CEL), pentosidine, pyrraline, FFI, AFGP and ALI. The AGE antigen may be an AGE-protein conjugate, such as AGE conjugated to keyhole limpet hemocyanin (AGE-KLH). Further details of some of these AGE-modified proteins or peptides and their preparation are described in Bucala.

Particularly preferred AGE antigens include proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation, and have been correlated with juvenile genetic disorders. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents).

AGE antigens may be conjugated to carrier proteins to enhance antibody production in a subject. Antigens that are not sufficiently immunogenic alone may require a suitable carrier protein to stimulate a response from the immune system. Examples of suitable carrier proteins include keyhole limpet hemocyanin (KLH), serum albumin, bovine thyroglobulin, cholera toxin, labile enterotoxin, silica particles and soybean trypsin inhibitor. Preferably, the carrier protein is KLH (AGE-KLH). KLH has been extensively studied and has been identified as an effective carrier protein in experimental cancer vaccines. Preferred AGE antigen-carrier protein conjugates include CML-KLH and CEL-KLH.

The administration of an AGE antigen allows the immune system to develop immunity to the antigen. Immunity is a long-term immune response, either cellular or humoral. A cellular immune response is activated when an antigen is presented, preferably with a co-stimulator to a T-cell which causes it to differentiate and produce cytokines. The cells involved in the generation of the cellular immune response are two classes of T-helper (Th) cells, Th1 and Th2. Th1 cells stimulate B cells to produce predominantly antibodies of the IgG2A isotype, which activates the complement cascade and binds the Fc receptors of macrophages, while Th2 cells stimulate B cells to produce IgG1 isotype antibodies in mice, IgG4 isotype antibodies in humans, and IgE isotype antibodies. The human body also contains “professional” antigen-presenting cells such as dendritic cells, macrophages, and B cells.

A humoral immune response is triggered when a B cell selectively binds to an antigen and begins to proliferate, leading to the production of a clonal population of cells that produce antibodies that specifically recognize that antigen and which may differentiate into antibody-secreting cells, referred to as plasma-cells or memory-B cells. Antibodies are molecules produced by B-cells that bind a specific antigen. The antigen-antibody complex triggers several responses, either cell-mediated, for example by natural killers (NK) or macrophages, or serum-mediated, for example by activating the complement system, a complex of several serum proteins that act sequentially in a cascade that result in the lysis of the target cell.

Immunological adjuvants (also referred to simply as “adjuvants”) are the component(s) of a vaccine which augment the immune response to the immunogenic agent. Adjuvants function by attracting macrophages to the immunogenic agent and then presenting the agent to the regional lymph nodes to initiate an effective antigenic response. Adjuvants may also act as carriers themselves for the immunogenic agent. Adjuvants may induce an inflammatory response, which may play an important role in initiating the immune response.

Adjuvants include mineral compounds such as aluminum salts, oil emulsions, bacterial products, liposomes, immunostimulating complexes and squalene. Aluminum compounds are the most widely used adjuvants in human and veterinary vaccines. These aluminum compounds include aluminum salts such as aluminum phosphate (AlPO₄) and aluminum hydroxide (Al(OH)₃) compounds, typically in the form of gels, and are generically referred to in the field of vaccine immunological adjuvants as “alum.” Aluminum hydroxide is a poorly crystalline aluminum oxyhydroxide having the structure of the mineral boehmite. Aluminum phosphate is an amorphous aluminum hydroxyphosphate. Negatively charged species (for example, negatively charged antigens) can absorb onto aluminum hydroxide gels at neutral pH, whereas positively charged species (for example, positively charged antigens) can absorb onto aluminum phosphate gels at neutral pH. It is believed that these aluminum compounds provide a depot of antigen at the site of administration, thereby providing a gradual and continuous release of antigen to stimulate antibody production. Aluminum compounds tend to more effectively stimulate a cellular response mediated by Th2, rather than Th1 cells.

Emulsion adjuvants include water-in-oil emulsions (for example, Freund's adjuvants, such as killed mycobacteria in oil emulsion) and oil-in-water emulsions (for example, MF-59). Emulsion adjuvants include an immunogenic component, for example squalene (MF-59) or mannide oleate (Incomplete Freund's Adjuvants), which can induce an elevated humoral response, increased T cell proliferation, cytotoxic lymphocytes and cell-mediated immunity.

Liposomal or vesicular adjuvants (including paucilamellar lipid vesicles) have lipophilic bilayer domains and an aqueous milieu which can be used to encapsulate and transport a variety of materials, for example an antigen. Paucilamellar vesicles (for example, those described in U.S. Pat. No. 6,387,373) can be prepared by mixing, under high pressure or shear conditions, a lipid phase comprising a non-phospholipid material (for example, an amphiphile surfactant; see U.S. Pat. Nos. 4,217,344; 4,917,951; and 4,911,928), optionally a sterol, and any water-immiscible oily material to be encapsulated in the vesicles (for example, an oil such as squalene oil and an oil-soluble or oil-suspended antigen); and an aqueous phase such as water, saline, buffer or any other aqueous solution used to hydrate the lipids. Liposomel or vesicular adjuvants are believed to promote contact of the antigen with immune cells, for example by fusion of the vesicle to the immune cell membrane, and preferentially stimulate the Th1 sub-population of T-helper cells.

Other types of adjuvants include Mycobacterium bovis bacillus Calmette-Guérin (BCG), quill-saponin and unmethylated CpG dinucleotides (CpG motifs). Additional adjuvants are described in U.S. Patent Application Publication Pub. No. US 2010/0226932 (Sep. 9, 2010) and Jiang, Z-H. et aL “Synthetic vaccines: the role of adjuvants in immune targeting”, Current Medicinal Chemistry, Vol. 10(15), pp. 1423-39 (2003). Preferable adjuvants include Freund's complete adjuvant and Freund's incomplete adjuvant.

The vaccine may optionally include one or more preservatives, such as antioxidants, antibacterial and antimicrobial agents, as well as combinations thereof. Examples include benzethonium chloride, ethylenediamine-tetraacetic acid sodium (EDTA), thimerosal, phenol, 2-phenoxyethanol, formaldehyde and formalin; antibacterial agents such as amphotericin B, chlortetracycline, gentamicin, neomycin, polymyxin B and streptomycin; antimicrobial surfactants such as polyoxyethylene-9, 10-nonyl phenol (Triton N-101, octoxynol-9), sodium deoxycholate and polyoxyethylated octyl phenol (Triton X-I00). The production and packaging of the vaccine may eliminate the need for a preservative. For example, a vaccine that has been sterilized and stored in a sealed container may not require a preservative.

Other components of vaccines include pharmaceutically acceptable excipients, such as stabilizers, thickening agents, toxin detoxifiers, diluents, pH adjusters, tonicity adjustors, surfactants, antifoaming agents, protein stabilizers, dyes and solvents. Examples of such excipients include hydrochloric acid, phosphate buffers, sodium acetate, sodium bicarbonate, sodium borate, sodium citrate, sodium hydroxide, potassium chloride, potassium chloride, sodium chloride, polydimethylsilozone, brilliant green, phenol red (phenolsulfon-phthalein), glycine, glycerin, sorbitol, histidine, monosodium glutamate, potassium glutamate, sucrose, urea, lactose, gelatin, sorbitol, polysorbate 20, polysorbate 80 and glutaraldehyde. A variety of these components of vaccines, as well as adjuvants, are described in www.cdc.gov/vaccines/pubs/pinkbook/downloads/appendices/B/excipient-table-2.pdf and Vogel, F. R. et al., “A compendium of vaccine adjuvants and excipients”, Pharmaceutical Biotechnology, Vol. 6, pp. 141-228 (1995).

The vaccine may contain from 1 μg to 100 mg of at least one AGE antigen, including 10, 20, 30, 40, 50, 60, 70, 80, 90, 100, 200, 400, 800 or 1000 pg, or 2, 3, 4, 6, 7, 8, 9, 10, 20, 30, 40, 50, 60, 70, 80 or 90 mg. The amount used for a single injection corresponds to a unit dosage.

The vaccine may be provided in unit dosage form or in multidosage form, such as 2-100 or 2-10 doses. The unit dosages may be provided in a vial with a septum, or in a syringe with or without a needle. The vaccine may be administered intravenously, subdermally or intraperitoneally. Preferably, the vaccine is sterile.

The vaccine may be administered one or more times, such as 1 to 10 times, including 2, 3, 4, 5, 6, 7, 8 or 9 times, and may be administered over a period of time ranging from 1 week to 1 year, 2-10 weeks or 2-10 months. Furthermore, booster vaccinations may be desirable, over the course of 1 year to 20 years, including 2, 5, and 15 years.

A subject that receives a vaccine for AGE-modified proteins or peptides of a cell may be tested to determine if he or she has developed an immunity to the AGE-modified proteins or peptides. Suitable tests may include blood tests for detecting the presence of an antibody, such as immunoassays or antibody titers. An immunity to AGE-modified proteins or peptides may also be determined by monitoring the concentration and/or number of senescent cells over time. In addition to testing for the development of an immunity to AGE-modified proteins or peptides, a subject may also be tested to determine if the vaccination has been effective to treat kidney disease. A subject may be considered to have received an effective vaccination if he or she demonstrates an increase in GFR or eGFR between subsequent measurements or over time, or by measuring the concentration and/or number of senescent cells. Vaccination and subsequent testing may be repeated until the desired therapeutic result is achieved.

The vaccination process may be designed to provide immunity against multiple AGE moieties. A single AGE antigen may induce the production of AGE antibodies which are capable of binding to multiple AGE moieties. Alternatively, the vaccine may contain multiple AGE antigens. In addition, a subject may receive multiple vaccines, where each vaccine contains a different AGE antigen.

Any mammal with a kidney may be treated by the methods herein described. Humans are a preferred mammal for treatment. Other mammals that may be treated include mice, rats, goats, sheep, cows, horses and companion animals, such as dogs or cats. Alternatively, any of the mammals or subjects identified above may be excluded from the patient population in need of treatment for kidney disease.

A subject may be identified as in need of treatment based on a diagnosis with a kidney disease. Examples of kidney diseases that may be treated or prevented include chronic kidney disease (CKD), diabetic nephropathy, diabetic kidney disease, IgA nephropathy (also referred to as Berger's disease or synpharyngitic glomerulonephritis), Goodpasture's syndrome (also referred to as anti-glomerular basement membrane disease), minimal change disease, thin basement membrane disease, fibronectin glomerulopathy, nephrotic syndrome, Alport syndrome (hereditary nephritis), familial renal amyloidosis, lupus nephritis, Finnish-type nephrosis, acquired cystic kidney disease, pyelonephritis (kidney infection), medullary sponge kidney (Cacchi-Ricci disease), polycystic kidney disease, acute tubular necrosis, renal tubular acidosis, polycystic kidney disease, Fanconi syndrome, Bartter syndrome, Gitelman syndrome, Liddle's syndrome, interstitial nephritis, glomerulonephritis (including acute proliferative glomerulonephritis, endocapillary nephritis, mesangioproliferative glomerulonephritis, mesangiocapillary glomerulonephritis, membranoproliferative glomerulonephritis, acute crescentic glomerulonephritis, focal segmental glomerulonephritis, membranous glomerulonephritis, post-infectious glomerulonephritis, acute post-streptococcal glomerulonephritis and rapidly progressive glomerulonephritis) and glomerulosclerosis (including focal segmental glomerulosclerosis or FSGS, diabetic glomerulosclerosis and nodular glomerulosclerosis). Glomerulosclerosis is a preferred kidney disease for treatment.

Kidney disease may be diagnosed by measuring glomerular filtration rate (GFR) or estimated glomerular filtration rate (eGFR), urinalysis to detect protein or red blood cells in the urine, blood tests to measure waste products such as creatinine and urea nitrogen, renal imaging such as ultrasound or X-rays or a kidney biopsy. A preferred method of diagnosing kidney disease is GFR or eGFR.

A subject may also be identified as in need of treatment based on a diagnosis of one or more diseases or conditions that are known to cause kidney disease. Examples of diseases or conditions known to cause kidney disease include diabetes, systemic lupus erythematosus, cardiovascular disease, hypertension, cancer and infections such as influenza infection, coronavirus infection, strep throat, impetigo, bacterial endocarditis and HIV.

Subjects may also be identified as in need of treatment based on detection of advanced glycation end products in a sample obtained from the subject. Suitable samples include blood, skin, serum, saliva and urine. The diagnostic use of anti-AGE antibodies is discussed in more detail in International Patent Application Publication No. WO 2018/204679.

The treatments described herein may optionally be combined with known treatments for kidney disease such as administration of sevelamer, renal replacement therapy (for example, dialysis, hemodialysis, short daily dialysis, peritoneal dialysis, hemodiafiltration and hemofiltration) and kidney transplantation. The treatments may also optionally be combined with known methods for reducing AGEs such as reducing dietary AGE intake, administration of AGE inhibitors (for example, aminoguanidine, benfotiamine, pyridoxamine, OPB-9195 and AGE breakers) or using techniques to remove AGEs ex vivo (for example, cell separation processes such as magnetic cell separation and cellular purification processes, such as immunopanning and immunoadsorption). Reducing dietary AGE intake may be accomplished by cooking foods using low heat and high humidity cooking methods such as stewing, steaming, boiling, poaching and braising. Dietary AGE intake may also be reduced by limiting the consumption of foods that are high in AGEs (for example, fried foods, red meat, cheeses and animal fats) and/or increasing the consumption of foods that reduce AGEs (for example, brown rice and mushrooms).

The Present Application includes 66 nucleotide and amino acid sequences in the Sequence Listing filed herewith. Variants of the nucleotide and amino acid sequences are possible. Known variants include substitutions, deletions and additions to the sequences shown in SEQ ID NO: 4, 16 and 20. In SEQ ID NO: 4, the arginine (Arg or R) residue at position 128 may optionally be omitted. In SEQ ID NO: 16, the alanine residue at position 123 may optionally be replaced with a serine residue, and/or the tyrosine residue at position 124 may optionally be replaced with a phenylalanine residue. SEQ ID NO: 20 may optionally include the same substitutions as SEQ ID NO: 16 at positions 123 and 124. In addition, SEQ ID NO: 20 may optionally contain one additional lysine residue after the terminal valine residue.

### EXAMPLES

### Example 1: In Vivo Study of the Administration of Anti-Glycation End-Product Antibody

To examine the effects of an anti-glycation end-product antibody, the antibody was administered to the aged CD1(ICR) mouse (Charles River Laboratories), twice daily by intravenous injection, once a week, for three weeks (Days 1, 8 and 15), followed by a 10 week treatment-free period. The test antibody was a commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, MN; catalog no. MAB3247). A control reference of physiological saline was used in the control animals.

Mice referred to as “young” were 8 weeks old, while mice referred to as “old” were 88 weeks (±2 days) old. No adverse events were noted from the administration of the antibody. The different groups of animals used in the study are shown in Table 1.

TABLE 1
The different groups of animals used in the study

| | | | | Number of Animals | |
|---|---|---|---|---|---|
| | | | Dose Level | Main | Treatment- |
| Group | Test | | (μg/gm/BID/ | Study | Free |
| No. | Material | Mice | week) | Females | Females |
| 1 | Saline | young | 0 | 20 | — |
| 2 | Saline | old | 0 | 20 | 20 |
| 3 | Antibody | old | 2.5 | 20 | 20 |
| 4 | None | old | 0 | 20 | pre |
| 5 | Antibody | old | 5.0 | 20 | 20 |

— = Not Applicable, Pre = Subset of animals euthanized prior to treatment start for collection of adipose tissue.

p16^{Ink4a} mRNA, a marker for senescent cells, was quantified in adipose tissue of the groups by Real Time-qPCR. The results are shown in Table 2. In the table ΔΔCt=ΔCt mean control Group (2)−ΔCt mean experimental Group (1 or 3 or 5); Fold Expression=2^{-ΔΔCt}.

TABLE 2
P16^{INK4}ª mRNA quantified in adipose tissue

| | Group 2 vs | | Group 2 vs | | Group 2 vs | |
|---|---|---|---|---|---|---|
| Calculation | Group 1 | | Group 3 | | Group 5 | |
| (unadjusted to | Group | Group | Group | Group | Group | Group |
| Group 4: 5.59) | 2 | 1 | 2 | 3 | 2 | 5 |
| Mean ΔCt | 5.79 | 7.14 | 5.79 | 6.09 | 5.79 | 7.39 |

| | | | |
|---|---|---|---|
| ΔΔCt | −1.35 | −0.30 | −1.60 |
| Fold | 2.55 | 1.23 | 3.03 |
| Expression | | | |

The table above indicates that untreated old mice (Control Group 2) express 2.55-fold more p16^{Ink4a} mRNA than the untreated young mice (Control Group 1), as expected. This was observed when comparing Group 2 untreated old mice euthanized at end of recovery Day 85 to Group 1 untreated young mice euthanized at end of treatment Day 22. When results from Group 2 untreated old mice were compared to results from Group 3 treated old mice euthanized Day 85, it was observed that p16^{Ink4a} mRNA was 1.23-fold higher in Group 2 than in Group 3. Therefore, the level of p16^{Ink4a} mRNA expression was lower when the old mice were treated with 2.5 μg/gram/BID/week of antibody.

When results from Group 2 (Control) untreated old mice were compared to results from Group 5 (5 μg/gram) treated old mice euthanized Day 22, it was observed that p16^{Ink4a} mRNA was 3.03-fold higher in Group 2 (controls) than in Group 5 (5 μg/gram). This comparison indicated that the Group 5 animals had lower levels of p16^{Ink4a} a mRNA expression when they were treated with 5.0 μg/gram/BID/week, providing p16^{Ink4a} a mRNA expression levels comparable to that of the young untreated mice (i.e. Group 1). Unlike Group 3 (2.5 μg/gram) mice that were euthanized at end of recovery Day 85, Group 5 mice were euthanized at end of treatment Day 22.

These results indicate the antibody administration resulted in the killing of senescent cells.

The mass of the gastrocnemius muscle was also measured, to determine the effect of antibody administration on sarcopenia. The results are provided in Table 3. The results indicate that administration of the antibody increased muscle mass as compared to controls, but only at the higher dosage of 5.0 μg/gm/BID/week.

TABLE 3
Effect of antibody administration
on mass of the gastrocnemius muscle

| | | Absolute | Weight relative to |
|---|---|---|---|
| | | weight of | body mass of |
| | Summary | Gastrocnemius | Gastrocnemius |
| Group | Information | Muscle (g) | Muscle (%) |
| 1 | Mean | 0.3291 | 1.1037 |
| | SD | 0.0412 | 0.1473 |
| | N | 20 | 20 |
| 2 | Mean | 0.3304 | 0.7671 |
| | SD | 0.0371 | 0.1246 |
| | N | 20 | 20 |
| 3 | Mean | 0.3410 | 0.7706 |
| | SD | 0.0439 | 0.0971 |
| | N | 19 | 19 |
| 5 | Mean | 0.4074 | 0.9480 |
| | SD | 0.0508 | 0.2049 |
| | N | 9 | 9 |

These results demonstrate that administration of antibodies that bind to AGEs of a cell resulted in a reduction of cells expressing p16^{Ink4a}, a biomarker of senescence. The data show that reducing senescent cells leads directly to an increase in muscle mass in aged mice. These results indicate that the loss of muscle mass, a classic sign of sarcopenia, can be treated by administration of antibodies that bind to AGEs of a cell. The results suggest that administration of the antibodies would be effective in treating or preventing the onset of diabetes and diabetic complications by removing senescent cells.

### Example 2: Affinity and Kinetics of Test Antibody

The affinity and kinetics of the test antibody used in Example 1 were analyzed using Nα,Nα-bis(carboxymethyl)-L-lysine trifluoroacetate salt (Sigma-Aldrich, St. Louis, MO) as a model substrate for an AGE-modified protein of a cell. Label-free interaction analysis was carried out on a BIACORE™ T200 (GE Healthcare, Pittsburgh, PA), using a Series S sensor chip CM5 (GE Healthcare, Pittsburgh, PA), with Fc1 set as blank, and Fc2 immobilized with the test antibody (molecular weigh of 150,000 Da). The running buffer was a HBS-EP buffer (10 mM HEPES, 150 mM NaCl, 3 mM EDTA and 0.05% P-20, pH of 7.4), at a temperature of 25° C. Software was BIACORE™ T200 evaluation software, version 2.0. A double reference (Fc2-1 and only buffer injection), was used in the analysis, and the data was fitted to a Langmuir 1:1 binding model.

TABLE 4
Experimental set-up of affinity and kinetics analysis
Association and dissociation

| Flow path | Fc1 and Fc2 |
|---|---|
| Flow rate (μl/min.) | 30 |
| Association time (s) | 300 |
| Dissociation time (s) | 300 |
| Sample concentration (μM) | 20 − 5 − 1.25 (×2) − 0.3125 − 0.078 − 0 |

A graph of the response versus time is illustrated in FIG. 1. The following values were determined from the analysis: kₐ(1/Ms)=1.857×10³; k_{d}(1/s)=6.781×10⁻³; K_{D}(M)=3.651×10⁻⁶; Rₘₐₓ(RU)=19.52; and Chi²=0.114. Because the Chi² value of the fitting is less than 10% of Rₘₐₓ, the fit is reliable.

### Example 3: Construction and Production Of Murine Anti-AGE IgG2b Antibody and Chimeric Anti-AGE IgG1 Antibody

Murine and chimeric human anti-AGE antibodies were prepared. The DNA sequence of murine anti-AGE antibody IgG2b heavy chain is shown in SEQ ID NO: 12. The DNA sequence of chimeric human anti-AGE antibody IgG1 heavy chain is shown in SEQ ID NO: 13. The DNA sequence of murine anti-AGE antibody kappa light chain is shown in SEQ ID NO: 14. The DNA sequence of chimeric human anti-AGE antibody kappa light chain is shown in SEQ ID NO: 15. The gene sequences were synthesized and cloned into high expression mammalian vectors. The sequences were codon optimized. Completed constructs were sequence confirmed before proceeding to transfection.

HEK293 cells were seeded in a shake flask one day before transfection, and were grown using serum-free chemically defined media. The DNA expression constructs were transiently transfected into 0.03 liters of suspension HEK293 cells. After 20 hours, cells were sampled to obtain the viabilities and viable cell counts, and titers were measured (Octet QKe, ForteBio). Additional readings were taken throughout the transient transfection production runs. The cultures were harvested on day 5, and an additional sample for each was measured for cell density, viability and titer.

The conditioned media for murine and chimeric anti-AGE antibodies were harvested and clarified from the transient transfection production runs by centrifugation and filtration. The supernatants were run over a Protein A column and eluted with a low pH buffer. Filtration using a 0.2 pm membrane filter was performed before aliquoting. After purification and filtration, the protein concentrations were calculated from the OD280 and the extinction coefficient. A summary of yields and aliquots is shown in Table 5:

TABLE 5
Yields and aliquots

| | Concentration | Volume | No. of | Total Yield |
|---|---|---|---|---|
| Protein | (mg/mL) | (mL) | vials | (mg) |
| Murine anti-AGE | 0.08 | 1.00 | 3 | 0.24 |
| Chimeric anti-AGE | 0.23 | 1.00 | 3 | 0.69 |

Antibody purity was evaluated by capillary electrophoresis sodium-dodecyl sulfate (CE-SDS) analysis using LabChip® GXII, (PerkinElmer).

### Example 4: Binding of Murine (Parental) and Chimeric Anti-AGE Antibodies

The binding of the murine (parental) and chimeric anti-AGE antibodies described in Example 3 was investigated by a direct binding ELISA. An anti-carboxymethyl lysine (CML) antibody (R&D Systems, MAB3247) was used as a control. CML was conjugated to KLH (CML-KLH) and both CML and CML-KLH were coated overnight onto an ELISA plate. HRP-goat anti-mouse Fc was used to detect the control and murine (parental) anti-AGE antibodies. HRP-goat anti-human Fc was used to detect the chimeric anti-AGE antibody.

The antigens were diluted to 1 μg/mL in 1× phosphate buffer at pH 6.5. A 96-well microtiter ELISA plate was coated with 100 pUwell of the diluted antigen and let sit at 4° C. overnight. The plate was blocked with 1× PBS, 2.5% BSA and allowed to sit for 1-2 hours the next morning at room temperature. The antibody samples were prepared in serial dilutions with 1× PBS, 1% BSA with the starting concentration of μg/mL. Secondary antibodies were diluted 1:5,000. 100 μL of the antibody dilutions was applied to each well. The plate was incubated at room temperature for hour on a microplate shaker. The plate was washed 3 times with 1× PBS. 100 μL/well diluted HRP-conjugated goat anti-human Fc secondary antibody was applied to the wells. The plate was incubated for 1 hour on a microplate shaker. The plate was then washed 3 times with 1× PBS. 100 μL HRP substrate TMB was added to each well to develop the plate. After 3-5 minutes elapsed, the reaction was terminated by adding 100 μL of 1N HCl. A second direct binding ELISA was performed with only CML coating. The absorbance at OD450 was read using a microplate reader.

The OD450 absorbance raw data for the CML and CML-KLH ELISA is shown in the plate map below. 48 of the 96 wells in the well plate were used. Blank wells in the plate map indicate unused wells.

Plate map of CML and CML-KLH ELISA:

| Conc. | | | | | | | |
|---|---|---|---|---|---|---|---|
| (μg/mL) | 1 | 2 | 3 | 4 | 5 | 6 | 7 |
| 50 | 0.462 | 0.092 | 0.42 | | 1.199 | 0.142 | 1.852 |
| 16.67 | 0.312 | 0.067 | 0.185 | | 0.31 | 0.13 | 0.383 |
| 5.56 | 0.165 | 0.063 | 0.123 | | 0.19 | 0.115 | 0.425 |
| 1.85 | 0.092 | 0.063 | 0.088 | | 0.146 | 0.099 | 0.414 |
| 0.62 | 0.083 | 0.072 | 0.066 | | 0.108 | 0.085 | 0.248 |
| 0.21 | 0.075 | 0.066 | 0.09 | | 0.096 | 0.096 | 0.12 |
| 0.07 | 0.086 | 0.086 | 0.082 | | 0.098 | 0.096 | 0.098 |
| 0 | 0.09 | 0.085 | 0.12 | | 0.111 | 0.083 | 0.582 |
| | R&D | Parental | Chimeric | | R&D | Parental | Chimeric |
| | Positive | Anti- | Anti- | | Positive | Anti- | Anti- |
| | Control | AGE | AGE | | Control | AGE | AGE |

| | |
|---|---|
| CML-KLH Coat | CML Coat |

The OD450 absorbance raw data for the CML-only ELISA is shown in the plate map below. 24 of the 96 wells in the well plate were used. Blank wells in the plate map indicate unused wells.

Plate map of CML-only ELISA:

| Conc. | | | | | | | |
|---|---|---|---|---|---|---|---|
| (μg/mL) | 1 | 2 | 3 | 4 | 5 | 6 | 7 |
| 50 | 1.913 | 0.165 | 0.992 | | | | |
| 16.66667 | 1.113 | 0.226 | 0.541 | | | | |
| 5.555556 | 0.549 | 0.166 | 0.356 | | | | |
| 1.851852 | 0.199 | 0.078 | 0.248 | | | | |
| 0.617284 | 0.128 | 0.103 | 0.159 | | | | |
| 0.205761 | 0.116 | 0.056 | 0.097 | | | | |
| 0.068587 | 0.073 | 0.055 | 0.071 | | | | |
| 0 | 0.053 | 0.057 | 0.06 | | | | |
| | R&D | Parental | Chimeric | | | | |
| | Positive | Anti- | Anti- | | | | |
| | Control | AGE | AGE | | | | |

The control and chimeric anti-AGE antibodies showed binding to both CML and CML-KLH. The murine (parental) anti-AGE antibody showed very weak to no binding to either CML or CML-KLH. Data from repeated ELISA confirms binding of the control and chimeric anti-AGE to CML. All buffer control showed negative signal.

### Example 5: Humanized Antibodies

Humanized antibodies were designed by creating multiple hybrid sequences that fuse select parts of the parental (mouse) antibody sequence with the human framework sequences. Acceptor frameworks were identified based on the overall sequence identity across the framework, matching interface position, similarly classed CDR canonical positions, and presence of N-glycosylation sites that would have to be removed. Three humanized light chains and three humanized heavy chains were designed based on two different heavy and light chain human acceptor frameworks. The amino acid sequences of the heavy chains are shown in SEQ ID NO: 29, 31 and 33, which are encoded by the DNA sequences shown in SEQ ID NO: 32 and 34, respectively. The amino acid sequences of the light chains are shown in SEQ ID NO: 35, 37 and 39, which are encoded by the DNA sequences shown in SEQ ID NO: 36, 38 and 40, respectively. The humanized sequences were methodically analyzed by eye and computer modeling to isolate the sequences that would most likely retain antigen binding. The goal was to maximize the amount of human sequence in the final humanized antibodies while retaining the original antibody specificity. The light and heavy humanized chains could be combined to create nine variant fully humanized antibodies.

The three heavy chains and three light chains were analyzed to determine their humanness. Antibody humanness scores were calculated according to the method described in Gao, S. H., et al., “Monoclonal antibody humanness score and its applications”, BMC Biotechnology, 13:55 (Jul. 5, 2013). The humanness score represents how human-like an antibody variable region sequence looks. For heavy chains a score of 79 or above is indicative of looking human-like; for light chains a score of 86 or above is indicative of looking human-like. The humanness of the three heavy chains, three light chains, a parental (mouse) heavy chain and a parental (mouse) light chain are shown below in Table 6:

TABLE 6
Antibody humanness

| | Humanness |
|---|---|
| Antibody | (Framework + CDR) |
| Parental (mouse) heavy chain | 63.60 |
| Heavy chain 1 (SEQ ID NO: 29) | 82.20 |
| Heavy chain 2 (SEQ ID NO: 31) | 80.76 |
| Heavy chain 3 (SEQ ID NO: 33) | 81.10 |
| Parental (mouse) light chain | 77.87 |
| Light chain 1 (SEQ ID NO: 35) | 86.74 |
| Light chain 2 (SEQ ID NO: 37) | 86.04 |
| Light chain 3 (SEQ IN NO: 39) | 83.57 |

Full-length antibody genes were constructed by first synthesizing the variable region sequences. The sequences were optimized for expression in mammalian cells. These variable region sequences were then cloned into expression vectors that already contain human Fc domains; for the heavy chain, the IgG1 was used.

Small scale production of humanized antibodies was carried out by transfecting plasmids for the heavy and light chains into suspension HEK293 cells using chemically defined media in the absence of serum. Whole antibodies in the conditioned media were purified using MabSelect SuRe Protein A medium (GE Healthcare).

Nine humanized antibodies were produced from each combination of the three heavy chains having the amino acid sequences shown in SEQ ID NO: 29, 31 and 33 and three light chains having the amino acid sequences shown in SEQ ID NO: 35, 37 and 39. A comparative chimeric parental antibody was also prepared. The antibodies and their respective titers are shown below in Table 7:

TABLE 7
Antibody titers

| Antibody | Titer (mg/L) |
|---|---|
| Chimeric parental | 23.00 |
| SEQ ID NO: 29 + SEQ ID NO: 35 | 24.67 |
| SEQ ID NO: 29 + SEQ ID NO: 37 | 41.67 |
| SEQ ID NO: 29 + SEQ ID NO: 39 | 29.67 |
| SEQ ID NO: 31 + SEQ ID NO: 35 | 26.00 |
| SEQ ID NO: 31 + SEQ ID NO: 37 | 27.33 |
| SEQ ID NO: 31 + SEQ ID NO: 39 | 35.33 |
| SEQ ID NO: 33 + SEQ ID NO: 35 | 44.00 |
| SEQ ID NO: 33 + SEQ ID NO: 37 | 30.33 |
| SEQ ID NO: 33 + SEQ ID NO: 39 | 37.33 |

The binding of the humanized antibodies may be evaluated, for example, by dose-dependent binding ELISA or cell-based binding assay.

### Example 6 (Prophetic): An AGE-RNAse Containing Vaccine in a Human Subject.

AGE-RNAse is prepared by incubating RNAse in a phosphate buffer solution containing 0.1-3 M glucose, glucose-6-phosphate, fructose or ribose for 10-100 days. The AGE-RNAse solution is dialyzed and the protein content is measured. Aluminum hydroxide or aluminum phosphate, as an adjuvant, is added to 100 μg of the AGE-RNAse. Formaldehyde or formalin is added as a preservative to the preparation. Ascorbic acid is added as an antioxidant. The vaccine also includes phosphate buffer to adjust the pH and glycine as a protein stabilizer. The composition is injected intravenously into a subject with chronic kidney disease.

### Example 7 (Prophetic): Injection Regimen for an AGE-RNAse Containing Vaccine in a Human Subject.

The same vaccine as described in Example 6 is injected intravenously into a subject with glomerulosclerosis. The titer of antibodies to AGE-RNAse is determined by ELISA after two weeks. Additional injections are performed after three weeks and six weeks, respectively. Further titer determination is performed two weeks after each injection.

### Example 8 (Prophetic): An AGE-Hemoglobin Containing Vaccine in a Human Subject.

AGE-hemoglobin is prepared by incubating human hemoglobin in a phosphate buffer solution containing 0.1-3 M glucose, glucose-6-phosphate, fructose or ribose for 10-100 days. The AGE-hemoglobin solution is dialyzed and the protein content is measured. All vaccine components are the same as in Example 6, except AGE-hemoglobin is substituted for AGE-RNAse. Administration is carried out as in Example 6, or as in Example 7.

### Example 9 (Prophetic): An AGE-Human Serum Albumin Containing Vaccine in a Human Subject.

AGE-human serum albumin is prepared by incubating human serum albumin in a phosphate buffer solution containing 0.1-3 M glucose, glucose-6-phosphate, fructose or ribose for 10-100 days. The AGE-human serum albumin solution is dialyzed and the protein content is measured. All vaccine components are the same as in Example 6, except AGE-human serum albumin is substituted for AGE-RNAse. Administration is carried out as in Example 6, or as in Example 7.

Example 10: Carboxymethyllysine-Modified Protein Vaccine for a Human Subject (Prophetic)

A vaccine is prepared by combining a carboxymethyllysine-modified protein as an AGE antigen, aluminum hydroxide as an adjuvant, formaldehyde as a preservative, ascorbic acid as an antioxidant, a phosphate buffer to adjust the pH of the vaccine and glycine as a protein stabilizer. The vaccine is injected subcutaneously into a subject with diabetic nephropathy.

### Example 11: Carboxyethyllysine-Modified Peptide Vaccine for a Human Subject (Prophetic)

A vaccine is prepared by combining a carboxyethyllysine-modified peptide conjugated to KLH as an AGE antigen, aluminum hydroxide as an adjuvant, formaldehyde as a preservative, ascorbic acid as an antioxidant, a phosphate buffer to adjust the pH of the vaccine and glycine as a protein stabilizer. The vaccine is injected subcutaneously into a subject with glomerulonephritis.

### Example 12: In Vivo Study of the Administration of a Carboxymethyl Lysine Monoclonal Antibody

The effect of a carboxymethyl lysine antibody on tumor growth, metastatic potential and cachexia was investigated. In vivo studies were carried out in mice using a murine breast cancer tumor model. Female BALB/c mice (BALB/cAnNCrl, Charles River) were eleven weeks old on Day 1 of the study.

4T1 murine breast tumor cells (ATCC CRL-2539) were cultured in RPMI 1640 medium containing 10% fetal bovine serum, 2 mM glutamine, 25 μg/mL gentamicin, 100 units/mL penicillin G Na and 100 μg/mL streptomycin sulfate. Tumor cells were maintained in tissue culture flasks in a humidified incubator at 37° C. in an atmosphere of 5% CO₂ and 95% air.

The cultured breast cancer cells were then implanted in the mice. 4T1 cells were harvested during log phase growth and re-suspended in phosphate buffered saline (PBS) at a concentration of 1×10⁶ cells/mL on the day of implant. Tumors were initiated by subcutaneously implanting 1×10⁵ 4T1 cells (0.1 mL suspension) into the right flank of each test animal. Tumors were monitored as their volumes approached a target range of 80-120 mm³. Tumor volume was determined using the formula: tumor volume=(tumor width)² (tumor length)/2. Tumor weight was approximated using the assumption that 1 mm³ of tumor volume has a weight of 1 mg. Thirteen days after implantation, designated as Day 1 of the study, mice were sorted into four groups (n=15/group) with individual tumor volumes ranging from 108 to 126 mm³ and a group mean tumor volume of 112 mm³. The four treatment groups are shown in Table 8 below:

TABLE 8
Treatment groups

| | | | Dosing |
|---|---|---|---|
| Group | Description | Agent | (μg/g) |
| 1 | Control | phosphate buffered saline (PBS) | N/A |
| 2 | Low-dose | carboxymethyl lysine monoclonal | 5 |
| | | antibody | |
| 3 | High-dose | carboxymethyl lysine monoclonal | 10 |
| | | antibody | |
| 4 | Observation | None | N/A |
| | only | | |

An anti-carboxymethyl lysine monoclonal antibody was used as a therapeutic agent. 250 mg of carboxymethyl lysine monoclonal antibody was obtained from R&D Systems (Minneapolis, MN). Dosing solutions of the carboxymethyl lysine monoclonal antibody were prepared at 1 and 0.5 mg/mL in a vehicle (PBS) to provide the active dosages of 10 and 5 μg/g, respectively, in a dosing volume of 10 mL/kg. Dosing solutions were stored at 4° C. protected from light.

All treatments were administered intravenously (i.v.) twice daily for 21 days, except on Day 1 of the study where the mice were administered one dose. On Day 19 of the study, i.v. dosing was changed to intraperitoneal (i.p.) dosing for those animals that could not be dosed i.v. due to tail vein degradation. The dosing volume was 0.200 mL per 20 grams of body weight (10 mL/kg), and was scaled to the body weight of each individual animal.

The study continued for 23 days. Tumors were measured using calipers twice per week. Animals were weighed daily on Days 1-5, then twice per week until the completion of the study. Mice were also observed for any side effects. Acceptable toxicity was defined as a group mean body weight loss of less than 20% during the study and not more than 10% treatment-related deaths. Treatment efficacy was determined using data from the final day of the study (Day 23).

The ability of the anti-carboxymethyl lysine antibody to inhibit tumor growth was determined by comparing the median tumor volume (MTV) for Groups 1-3. Tumor volume was measured as described above. Percent tumor growth inhibition (% TGI) was defined as the difference between the MN of the control group (Group 1) and the MTV of the drug-treated group, expressed as a percentage of the MN of the control group. % TGI may be calculated according to the formula: % TGI=(1−MTV_{treated}/MTV_{control})×100.

The ability of the anti-carboxymethyl lysine antibody to inhibit cancer metastasis was determined by comparing lung cancer foci for Groups 1-3. Percent inhibition (% Inhibition) was defined as the difference between the mean count of metastatic foci of the control group and the mean count of metastatic foci of a drug-treated group, expressed as a percentage of the mean count of metastatic foci of the control group. % Inhibition may be calculated according to the following formula:

% Inhibition=(1−Mean Count of Foci_{treated}/Mean Count of Foci_{control})×100.

The ability of the anti-carboxymethyl lysine antibody to inhibit cachexia was determined by comparing the weights of the lungs and gastrocnemius muscles for Groups 1-3. Tissue weights were also normalized to 100 g body weight.

Treatment efficacy was also evaluated by the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study.

Statistical analysis was carried out using Prism (GraphPad) for Windows 6.07. Statistical analyses of the differences between Day 23 mean tumor volumes (MTVs) of two groups were accomplished using the Mann-Whitney U test. Comparisons of metastatic foci were assessed by ANOVA-Dunnett. Normalized tissue weights were compared by ANOVA. Two-tailed statistical analyses were conducted at significance level P=0.05. Results were classified as statistically significant or not statistically significant.

The results of the study are shown below in Table 9:

TABLE 9
Results

| | | | | | | | Gastroc. | Lung |
|---|---|---|---|---|---|---|---|---|
| | | | | | | | weight/ | weight/ |
| | MTV | | Lung | % | | | normalized | normalized |
| Group | (mm³) | % TGI | foci | Inhibition | PR | CR | (mg) | (mg) |
| 1 | 1800 | N/A | 70.4 | N/A | 0 | 10 | 353.4/19.68 | 2799.4/292.98 |
| 2 | 1568 | 13% | 60.3 | 14% | 0 | 0 | 330.4/21.62 | 2388.9/179.75 |
| 3 | 1688 | 6% | 49.0 | 30% | 0 | 0 | 398.6/24.91 | 2191.6/214.90 |

All treatment regimens were acceptably tolerated with no treatment-related deaths. The only animal deaths were non-treatment-related deaths due to metastasis. The % TGI trended towards significance (P>0.05, Mann-Whitney) for the 5 μg/g (Group 2) or 10 μg/g treatment group (Group 3). The % Inhibition trended towards significance (P>0.05, ANOVA-Dunnett) for the 5 μg/g treatment group. The % Inhibition was statistically significant (P≤0.01, ANOVA-Dunnett) for the 10 μg/g treatment group. The ability of the carboxymethyl lysine antibody to treat cachexia trended towards significance (P>0.05, ANOVA) based on a comparison of the organ weights of the lung and gastrocnemius between treatment groups and the control group. The results indicate that administration of an anti-carboxymethyl lysine monoclonal antibody is able to reduce cancer metastases. This data provides additional evidence that in vivo administration of anti-AGE antibodies can provide therapeutic benefits safely and effectively.

### Example 13: In Vivo Study of the Administration of Anti-Glycation End-Product Antibody (Prophetic)

An anti-glycation end-product antibody will be administered to the humanized mouse model of nephritis (lupus nephritis) (Gunawan, M., et al., “A novel human systematic lupus erythematosus model in humanized mice”, Scientific Reports, Vol. 7, pp. 1-11 (2017)). The antibody will be administered by intravenous injection, once a week, for three weeks (Days 1, 8 and 15). The test antibody will be a humanized anti-glycation end-product antibody raised against carboxymethyl lysine (anti-CML antibody). A control reference of physiological saline will be used in the control animals.

The different groups of animals that will be used in the study are shown in Table 10:

TABLE 10
The different groups of animals that will be used in the study

| | | | | Number of Animals | |
|---|---|---|---|---|---|
| | | | Dose Level | Main | Treatment- |
| Group | Test | | (μg/gm/BID/ | Study | Free |
| No. | Material | Mice | week) | Females | Females |
| 1 | Saline | young | 0 | 20 | — |
| 2 | Saline | old | 0 | 20 | 20 |
| 3 | Antibody | old | 5.0 | 20 | 20 |
| 4 | None | old | 0 | 20 | pre |
| 5 | Antibody | old | 10.0 | 20 | 20 |

— = Not Applicable, Pre = Subset of animals euthanized prior to treatment start for collection of adipose tissue.

p16^{Ink4a} mRNA, a marker for senescent cells, will be quantified in adipose tissue of the groups obtained after euthanasia by Real Time-qPCR. The treated mice will have a lower level of p16^{Ink4a} mRNA. A greater reduction in p16^{Ink4a} mRNA is expected at the 10.0 μg/gram dosage than the 5.0 μg/gram dosage. These results will indicate that administration of the anti-AGE antibody kills senescent cells.

Kidney function will be measured using GFR. The treated mice will have a higher GFR than the untreated mice. A greater GFR is expected at the 10.0 μg/gram dosage than the 5.0 μg/gram dosage. These results will indicate that administration of the anti-AGE antibody improves kidney function.

Kidney disease will also be evaluated by analyzing the glomeruli of the mice after euthanasia. The treated mice will exhibit fewer signs of glomerulosclerosis, as determined by detecting signs of fibrosis, such as scarring or hardening, in the glomeruli. A smaller degree of glomerulosclerosis is expected at the 10.0 μg/gram dosage than the 5.0 μg/gram dosage. These results will indicate that administration of the anti-AGE antibody treats or prevents glomerulosclerosis.

### Example 14: Primary Renal Tube Epithelial Cells Exposed to Hydrogen Peroxide

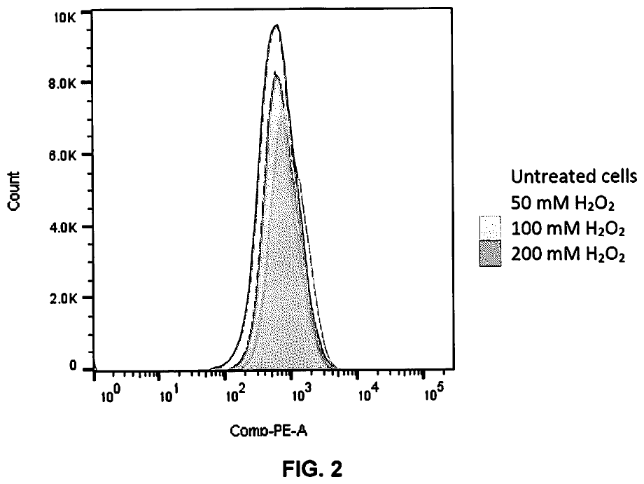

In vitro primary renal tube epithelial cells were exposed to different concentrations of hydrogen peroxide. Using a senescent cell assay kit, the number of senescent cells was detected. The increasing hydrogen peroxide concentrations are associated with an increase in the number of senescent cells. The data is shown in FIG. 2.

### Example 15: Antibody Assay (Prophetic)

First, the antibody assay will be optimized on frozen and paraffin-embedded tissues. “Normal” renal tissue will be used as a positive control in order to determine optimal staining conditions by varying antibody dilutions, incubation times, and antigen retrieval methods. Preferably, optimized staining conditions for paraffin-embedded tissues will be obtained. Once optimal conditions have been determined, a variety of renal tissues will be stained to determine their reactivity with 318H mAb. These will include kidney biopsies from individuals with: (a) diabetic nephropathy of varying severity; (b) all WHO classes of lupus nephritis; (c) renal cell carcinomas, of clear cell, papillary (cortical tubular epithelial origin), and chromophobe types, as well as oncocytomas (collecting duct origin): tissue microarrays constructed with multiple tumor specimens will be used for this purpose and will also contain cases of other non-renal tumors such as breast, pancreas, and colon adenocarcinomas; (d) acute kidney injury (AKI) from acute tubular necrosis (ATN); (e) COVID-associated renal disease; (f) kidney transplants, experiencing antibody-mediated rejection, cell-mediated rejection, transplant glomerulopathy, or polyoma virus nephropathy.

### Example 16: Human Embryonic Kidney (HEK) Cells

HEK-293 cells were transferred to a 96 well round bottom plate. Each well was centrifuged and cells washed with 200 μL PBS and subsequently suspended in 50 μL of 20 μg/mL 318H antibody conjugated with Texas Red. The samples were immediately incubated and protected from light, for one hour. At the end of the incubation period the samples were washed three times with FACS buffer (PBS supplemented with 1% fetal bovine serum) and immediately analyzed using flow cytometry. Flow cytometry analysis showed that the 318H antibody bound to the HEK cells. HEK cells are considered abnormal cells, similar to cancer cells. (Graham, F. L., et al., Characteristics of a Human Cell Line Transformed by DNA from Human Adenovirus Type 5, J. gen. Virol., vol. 36, pg. 59-74 (1977)). The fact that the 318H antibody binds to the HEK cells shows that the antibody can bind to cancer cells.

### REFERENCES

1. U.S. Pat. No. 10,526,579.
2. U.S. Patent Application Publication No. 20180344803.
3. U.S. Patent Application Publication No. 20180284131.
4. U.S. Patent Application Publication No. 20180252705.
5. U.S. Pat. No. 9,867,923.
6. U.S. Patent Application Publication No. 20170269060.
7. U.S. Patent Application Publication No. 20160304959.
8. U.S. Patent Application Publication No. 20160296592.
9. U.S. Patent Application Publication No. 20160169910.
10. U.S. Pat. No. 9,345,739.
11. U.S. Patent Application Publication No. 20150376575.
12. U.S. Pat. No. 9,205,135.
13. U.S. Pat. No. 9,144,594.
14. U.S. Patent Application Publication No. 20140302065.
15. U.S. Patent Application Publication No. 20140083945.
16. U.S. Pat. No. 8,668,927.
17. U.S. Patent Application Publication No. 20120251527.
18. U.S. Patent Application Publication No. 20120219542.
19. U.S. Patent Application Publication No. 20120213775.
20. U.S. Patent Application Publication No. 20120195876.
21. U.S. Patent Application Publication No. 20110236397
22. U.S. Patent Application Publication No. 20110212083.
23. U.S. Patent Application Publication No. 20100297139.
24. U.S. Patent Application Publication No. 20100272709.
25. U.S. Patent Application Publication No. 20100221317.
26. U.S. Pat. No. 7,670,817.
27. U.S. Patent Application Publication No. 20070197438.
28. Reiser, J., “A humanized mouse model of FSGS”, available online at grantome.com/grant/NIH/R01-DK106051-02 (2015).
29. Tanji, N. et al., “Expression of advanced glycation end products and their cellular receptor RAGE in diabetic nephropathy and nondiabetic renal disease”, Journal of the American Society of Nephrology, Vol. 11, pp. 1656-1666 (2000).
30. Wei, C. et al., “uPAR isoform 2 forms a dimer and induces severe kidney disease in mice”, The Journal of Clinical Investigation, Vol. 129, No. 5, pp.1946-1959 (2019).
31. Oldfield, M. D. et al., “Advanced glycation end products cause epithelial-myofibroblast transdifferentiation via the receptor for advanced glycation end products (RAGE)”, The Journal of Clinical Investigation, Vol. 108, No. 12, pp. 1853-1863 (2001).
32. Wei, C. et al., “Circulating CD40 autoantibody and suPAR synergy drives glomerular injury”, Annals of Translational Medicine, Vol. 3, No. 19, 5 pages (2015).
33. Suzuki, D. et al., “Immunohistochemical evidence for an increased oxidative stress and carbonyl modification of proteins in diabetic glomerular lesions”, Journal of the American Society of Nephrology, Vol. 10, pp. 822-832 (1999).
34. Horie, K. et al., “Immunohistochemical colocalization of glycoxidation products and lipid peroxidation products in diabetic renal glomerular lesions”, The Journal of Clinical Investigation, Vol. 100, No. 12, pp. 2995-3004 (1997).
35. Kushiro, M. et al., “Accumulation of N^{σ}-(carboxy-methyl)lysine and changes in glomerular extracellular matrix components in Otsuka Long-Evans Tokushima fatty rat: a model of spontaneous NIDDM”, Nephron, Vol. 79, No. 4, pp. 458-468 (1998).
36. Valentijn, F. A. et al., “Cellular senescence in the aging and diseased kidney”, Journal of Cell Communication and Signaling, Vol. 12, pp. 69-82 (2018).
37. Wang, W-J. et al., “Cellular senescence, senescence-associated secretory phenotype, and chronic kidney disease”, Oncotarget, Vol. 8, No. 38, pp. 64520-64533 (2017).
38. de Souza, V. A. et al., “Sarcopenia in patients with chronic kidney disease not yet on dialysis: analysis of the prevalence and associated factors”, PLoS ONE, Vol. 12, No. 4, e0176230, 13 pages (2017).
39. Semba, R. D. etal., “Serum carboxymethyl-lysine, a dominant advanced glycation end product, is associated with chronic kidney disease: the Baltimore longitudinal study of aging”, Journal of Renal Nutrition, Vol. 20, No. 2, pp. 74-81 (2010).
40. Uribarri, J. et al., “The low AGE diet: a neglected aspect of clinical nephrology practice?”, Nephron, Vol. 130, pp. 48-53 (2015).
41. “Chronic Kidney Disease Basics”, Centers for Disease Control and Prevention, available online at www.cdc.gov/kidneydisease/basics.html (2020).
42. “Chronic Kidney Disease”, World Kidney Day, available online at worldkidneyday.org/facts/chronic-kidney-disease/(2020).
43. Yang, C. et al., “Accelerated glomerular cell senescence in experimental lupus nephritis”, Medical Science Monitor, Vol. 24, pp. 6882-6891 (2018).
44. Xu, X., et aL, “A glimpse of matrix metalloproteinases in diabetic nephropathy”, Current Medicinal Chemistry, Vol. 21, No. 28, pp. 3244-3260 (2014).
45. Vas, T. et al., “Oxidative stress and non-enzymatic glycation in IgA nephropathy”, Clinical Nephrology, Vol. 64, No. 5, pp. 343-351 (2005).
46. Gunawan, M., et al., “A novel human systematic lupus erythematosus model in humanized mice”, Scientific Reports, Vol. 7, pp. 1-11 (2017).
47. Shenouda, F., et al., “Influenza A viral infection associated with acute renal failure”, Am. J. Med., Vol. 61, No. 5, pp. 697-702 (1976).
48. Graham, F.L., et al., Characteristics of a Human Cell Line Transformed by DNA from Human Adenovirus Type 5, J. gen. Virol., vol. 36, pg. 59-74 (1977).

1. A method of treating or preventing the onset of kidney disease, comprising administering to a subject, a composition comprising an anti-AGE antibody.

2. A method of treating or preventing the onset kidney disease, comprising administering to a subject a vaccine comprising an AGE antigen.

3. The method of claim 1, wherein the composition further comprises a second anti-AGE antibody.

4. The method of claim 1, wherein the composition further comprises a pharmaceutically acceptable carrier.

5. The method of claim 1, wherein the subject is selected from the group consisting of humans, goats, sheep, pigs, cows, horses, camels, alpacas, dogs and cats.

6. The method of claim 1, wherein the subject is a human.

7. The method of claim 1, wherein the anti-AGE antibody is non-immunogenic to a species selected from the group consisting of humans, cats, dogs, horses, camels, alpaca, cattle, sheep, pigs, and goats.

8. The method of claim 1, wherein the anti-AGE antibody binds an AGE antigen comprising at least one protein or peptide that exhibits AGE modifications selected from the group consisting of FFI, pyrraline, AFP, ALI, carboxymethyllysine, carboxyethyllysine and pentosidine.

9. The method of claim 1, wherein the anti-AGE antibody binds a carboxyrnethyllysine-modified protein or peptide.

10. The method of claim 1, wherein the composition is in unit dosage form.

11. The method of claim 1, wherein the composition is sterile.

12-17. (canceled)

18. The method of, further comprising testing the patient to determine if the kidney disease has been ameliorated, and
repeating the administering, if necessary.

19-21. (canceled)

22. The method of claim 1, wherein the antibody is a humanized antibody.

23. The method of claim 1, wherein the antibody is monoclonal.

24. The method of claim 1, wherein the antibody is substantially non-immunogenic to humans.

25 (canceled)

26. The method of claim 1, wherein the antibody is conjugated to an agent that causes the destruction of AGE-modified cells,
wherein the agent is selected from the group consisting of a toxin, a cytotoxic agent, magnetic nanoparticles, and magnetic spin-vortex discs.

27-28. (canceled)

29. The method of claim 1, wherein the kidney disease comprises at least one kidney disease selected from the group consisting of chronic kidney disease, diabetic nephropathy, diabetic kidney disease, IgA nephropathy, Goodpasture's syndrome, minimal change disease, thin basement membrane disease, fibronectin glomerulopathy, nephrotic syndrome, Alport syndrome, familial renal amyloidosis, lupus nephritis, Finnish-type nephrosis, acquired cystic kidney disease, pyelonephritis, medullary sponge kidney, polycystic kidney disease, acute tubular necrosis, renal tubular acidosis, polycystic kidney disease, Fanconi syndrome, Bartter syndrome, Gitelman syndrome, Liddle's syndrome, interstitial nephritis, glomerulonephritis, acute proliferative glomerulonephritis, endocapillary nephritis, mesangioproliferative glomerulonephritis, mesangiocapillary glomerulonephritis, membranoproliferative glomerulonephritis, acute crescentic glomerulonephritis, focal segmental glomerulonephritis, membranous glomerulonephritis, post-infectious glomerulonephritis, acute post-streptococcal glomerulonephritis, rapidly progressive glomerulonephritis, glomeruloscierosis, focal segmental glomerulosclerosis, diabetic glomerulosclerosis and nodular glomerulosclerosis.

30. The method of claim 1, wherein the kidney disease is glornerulosclerosis.

31-34. (canceled)

35. The method of claim 1, wherein the kidney disease is chronic kidney disease.

36. The method of claim 1, wherein the kidney disease is diabetic nephropathy.
